(19) United States
(12) Patent Application Publication
(10) Pub. No.: US 2006/0009798 A1
(43) Pub. Date: Jan. 12, 2006

(54) Methods and devices for occluding body lumens and/or enhancing tissue ingrowth

(75) Inventors: Jeffrey P. Callister, Redwood City, CA (US); William S. Tremulis, Redwood City, CA (US)

Correspondence Address: Edward J. Lynch;Duane Morris LLP, One Market, Spear Tower, 20th Floor, San Francisco, CA, 94105, US

(73) Assignee: AMS Research Corporation

(21) Appl. No.: 11/048,221

(22) Filed: Jan. 31, 2005

Related U.S. Application Data
(60) Provisional application No. 60/541,821, filed on Feb. 2, 2004.

(51) Int. Cl. A61F 2/01; A61F 2/04; A61F 2/84; A61F 2/88; A61F 2/90

(52) U.S. Cl. 606/200; 606/195; 623/1.16; 623/1.22; 623/23.7

(57) ABSTRACT

The present invention provides devices, methods and systems for the occlusion of various lumens in a body of a patient including devices and methods for enhancing tissue ingrowth, particularly endothelial tissue growth within an occlusive device. The system includes an occlusive device and a delivery device for placing the occlusive device in a body lumen. The occlusive device is generally a tubular member with a mesh member disposed thereon. The occlusive device is configured to be radially expandable along a longitudinal axis of the tubular member and implantable with a delivery catheter such that the occlusive device is in a collapsed state when positioned in the delivery catheter and in an expanded state when positioned in a lumen of a patient. The mesh member of the occlusive device is configured to promote epithelial tissue ingrowth.

### RELATED APPLICATIONS

This application claims priority to U.S. Provisional Application No. 60/541,821, filed Feb. 2, 2004, which is incorporated herein by reference in its entirety.

### BACKGROUND OF THE INVENTION

1. Field of the Invention

This invention relates to medical devices, methods and systems to implants for use in body lumens. More particularly, this invention relates to medical devices that are implanted within a body lumen (e.g., fallopian tube, vas deferens, bronchus, blood vessel, etc.) to occlude the body lumen and/or medical devices and methods that enhance tissue ingrowth.

2. Description of the Related Art

Surgical techniques, especially minimally invasive surgery (MIS) or percutaneous manipulation of body lumens and body passageways, have led to dramatic advances in medical treatments. For example, treatment of coronary heart disease has been greatly improved by percutaneous angioplasty and the subsequent or contemporaneous placement of stents in the coronary vasculature to open coronary arteries and maintain them open for blood flow. The placement of stents or similar devices surgically, by percutaneous methods, or with laprascopes, such as hysteroscopes, has been proposed for the purposes of obstructing body passageways. This technique may be advantageous for various purposes, but is of particular use in obstructing the fallopian tubes as a contraceptive alternative to tubal ligation.

Mere placement of a stent or similar device may not create sufficient or permanent obstruction of the body passageway depending on the nature of the occlusive device. For example, the occlusive device might be too small to create a complete obstruction of the body passageway, or might be slightly permeable. An occlusive device placed in the fallopian tubes, for example, might be too small to securely seal against the fallopian tube walls or might be formed of a screen like structure and thus be slightly permeable. It might create an obstruction sufficient to prevent the passage of an egg past the occlusive device, but might in fact be dangerous if sperm, a much smaller cell than the egg, is able to pass the obstruction and fertilize the egg upstream of the obstruction. In such a case, the fertilized egg would remain in the fallopian tube and an ectopic pregnancy could result.

Additionally, even if the occlusive device is large enough to fill the entire lumen, fallopian tubes tend to recannalize around an obstruction over time particularly if the obstruction is relatively inert or there is no tissue ingrowth. As with the incomplete obstruction, this may result in a passageway large enough for sperm to enter the upper portions of the fallopian tubes to fertilize an egg, but too small for the passage of an egg out of the fallopian tubes, and thus again an ectopic pregnancy might result.

One method addressing this problem, previously proposed by these inventors, is to enhance tissue ingrowth. Several methods have been disclosed, including inter alia, incorporating fibers or filaments on or within the occlusive devices. Additional devices and methods that provide enhanced passageway obstruction and encouraging tissue ingrowth or otherwise enhancing obstruction or contraception created by the device would be advantageous in various situations.

### SUMMARY OF THE INVENTION

The present invention provides devices, methods and systems for the occlusion of various passageways of the body including devices and methods that enhance the ingrowth of tissue and particularly endothelial tissue within the occlusive device. In the various aspects of occluding body passageways, particularly useful and immediate benefits for this invention are methods and systems for the delivery of occlusive devices to fallopian tubes for contraceptive purposes. Although occlusion of the fallopian tubes will be discussed in detail, it can be appreciated that the devices, methods and systems described herein can easily be adapted for other applications, for example, to occlude the vas in the male patient, arteries or veins in the nidus of an arterial-venous malformation, patent ductus arteriosis in infants, as well as feeding arteries to cancerous tumors, among other passageways.

The present invention also provides means for delivering vessel-supporting devices such as coronary stents or venous or arterial embolic filters, to the desired location through a steerable system. Another embodiment provides for delivery of therapeutic substances to desired locations and in advantageous manners. In some embodiments, this invention also provides for methods and devices that encourage tissue ingrowth to enhance the occlusion of the body lumen by the occlusive device.

In one embodiment of the present invention, the occlusive device is a tubular member and a mesh disposed on or in the tubular member to encourage tissue ingrowth, and the tissue ingrowth is further enhanced by electrical stimulation or electrical irritation created by galvanic action. The tubular member will be at least in part expandable within a body lumen from a first configuration suitable for insertion into the chosen location in the body lumen, to a second configuration larger in diameter than the first configuration to facilitate fastening the tubular member to the wall of the body lumen and creating an obstruction to block the body lumen.

In one embodiment, the tubular member is a stent-like structure expandable by a balloon catheter. The stent-like structure is mounted on the uninflated balloon of the catheter, and the catheter is introduced into the body lumen and placed with the balloon in the location where the user desires to place the occlusive device. The balloon is then inflated to the desired size, generally the diameter of the body lumen, which expands the stent-like structure out and against the sides of the body lumen, The balloon is then deflated and the catheter withdrawn, leaving the stent-like structure in place against the walls of the body structure.

In another embodiment, the tubular member may be a self-expanding structure, for example a structure with the outer portion constructed of a heat expandable metal. The device in one, small diameter configuration at one temperature is placed into the desired location, and the device is either heated by application, for example, of radio frequency energy, or attains a second temperature by being heated by the patient's body heat, and expands to the desired larger diameter configuration.

The occlusive device may be an open, lattice-like framework that may be secured in place in part by tissue ingrowth. The tissue ingrowth may also enhance the obstructive effect of the device, assisting to seal the body lumen. Fibers, mesh or the like may be contained in or on the occlusive device to encourage the tissue ingrowth.

Various means of constructing occlusive devices to generate galvanic action are anticipated by this invention. In general, if two different metal elements are insulated from each other and each is immersed in an electrolyte, and the two metals are connected by a conductor, current will flow from one element to the other. If the “conductor” is in fact that the two elements are in electrical contact, the current flow will be immediate and there will generally by corrosion or electrical irritation at the location of the contact between the two metals. The body fluid in which the occlusive device is immersed will generally suffice to create the galvanic action of the minimal magnitude required for these purposes.

Although this list is by no means exhaustive, some examples include: the construction of a stent using two different types of metal wire woven into an expandable structure; the creation of a stent like tube constructed of two different types of wire where one is a self expanding metal such as nitinol; the construction of an occlusive device made of one type of metal, with fibers inside the device to encourage ingrowth, where the fibers are metal fibers of a different type than the structure of the outside of the occlusive device; the construction of an occlusive device, perhaps a stent-like tube, with a metal wire or rod like metal element contained within the stent-like structure, perhaps running lengthwise down the tube, where the wire and the outside stent-like structure are constructed of different metals; and an occlusive device constructed of two helical coil springs made of different metals, with one of the springs contained within the other. In this last example the outer spring may be a self-expanding metal such as nitinol. The use of copper as one of the metals, whether as one of the elements of the outer occlusive device, the inner wire or rod, or the inner fiber may enhance the contraceptive action of the occlusive device if it is used for contraceptive purposes.

A method of relieving friction to provide for smooth delivery of an occlusive device through a delivery catheter is provided in one embodiment of the present invention. Another embodiment provides a tissue abrading catheter tip that further encourages tissue ingrowth. Yet another embodiment provides immediately effective devices for immediate obstruction of a body lumen even prior to the completion of tissue ingrowth. Other aspects of the present invention include a sealable end on a delivery catheter, a segmented occlusive device, and an expanding inner matrix.

The present invention provides devices, methods and systems for the occlusion of various body lumens. It also includes catheter systems for the delivery of such devices and systems. Typically these devices are delivered either by direct placement or by using “over-the-wire” (OTW) designs or techniques. The placement may be made directly or with the use of another device, for example a hysteroscope or other type of laparoscope. Although OTW designs allow for steerability of the guide wires and delivery catheters, the devices typically must have in inner diameter larger than the removable guide wire with which it is used. The diameter of the guide wire, however, may be too large, even it its smallest functional diameter, to allow for a small enough collapsed profile to transverse through the target passageway. The alternative means of using a pushing device proximal to the collapsed device allows for the device to have a very small collapsed profile since no guide wire needs to pass through it, however such systems may have reduced steerability of the system through the body lumens, particularly distal to the collapsed device. For these reasons and others it would be desirable to have a small diameter system that still allows for steerability of the guide wire while advancing through the body passageways.

Those skilled in the art will recognize that various combinations, modifications alternative embodiments may be equivalents and may be included in the invention without departing from the scope of the invention as set forth herein. For example, other acceptable variations of the delivery devices and the occlusive devices are disclosed in patent application Ser. Nos. 08/770,123, 09/112,085, 09/468,749 and Provisional Application Ser. No. 60/483,587, the complete contents of which are incorporated as if set forth in full herein.

### BRIEF DESCRIPTION OF THE DRAWINGS

FIG. 1A is a side elevational view of the occlusive device of the present invention in a collapsed state.

FIG. 1B is a side elevational view of the occlusive device of the present invention in an expanded state.

FIG. 2 is a side elevational view of the occlusive device of the present invention positioned within a body lumen.

FIG. 3 is a side elevational view of another embodiment of the occlusive device of the present invention having polyester fibers attached to an expandable frame.

FIG. 4 is a side perspective view of the delivery device of the present invention having two occlusive devices therein.

FIG. 5 is a front elevational view of two occlusive devices of the present invention implanted in two fallopian tubes.

FIG. 6A is an exploded perspective view of a delivery device and the occlusive device of the present invention.

FIG. 6B is an exploded perspective view of another aspect of the delivery device and occlusive device of the present invention.

FIG. 7 is an exploded perspective view of another aspect of the delivery device and the occlusive device of the present invention.

FIG. 8 is a side elevational view of the delivery device of the present invention having the occlusive device therein positioned on a steerable guidewire.

FIG. 9 is a side elevational view of another aspect of the delivery device depicted in FIG. 8.

FIG. 10A is a side elevational view of the occlusive device of the present invention in a collapsed state within the delivery device.

FIG. 10B is a side elevational view of the occlusive device of the present invention in an expanded state within a body lumen.

FIG. 10C is a side elevational view of another aspect of the occlusive device of the present invention in an expanded state within a body lumen.

FIG. 11 is a side elevational view of one embodiment of fibers comprising the occlusive device of the present invention.

FIG. 11A is an enlarged view of the fibers depicted in FIG. 11.

FIG. 11B is an enlarged view of another aspect of the fibers depicted in FIG. 11.

FIG. 12 is a side elevational view of the occlusive device of the present invention.

FIG. 12A is an enlarged view of one aspect of the occlusive device depicted in FIG. 12.

FIG. 12B is an enlarged view of another aspect of the occlusive device depicted in FIG. 12.

FIG. 12C is an enlarged view of a drug eluting container attached to the occlusive device of the present invention as depicted in FIG. 12.

FIG. 13 is an elevational view of the occlusive device of the present invention used with a separate substance delivering implantable intraluminal device where the occlusive device is shown in both a collapsed state and an expanded state.

FIG. 14 is a side elevational view of another embodiment of the delivery device of the present invention having visual markings and depth markers along a shaft of the delivery device and a flare at the distal end of the delivery shaft.

FIG. 15A is an elevational view of a flag placed on the distal end of the occlusive device of the present invention for determining the position of the device.

FIG. 15B is an elevational view of another embodiment of the flag placed on the distal of the occlusive device of the present invention.

FIG. 16 is a side elevational view of the occlusive device of the present invention implanted in a feeding artery of a tumor.

FIG. 17 is a side elevational view of the occlusive device of the present invention implanted in a bronchus of a lung.

FIG. 18 is an elevational view of one embodiment of the occlusive device of the invention with the tubular member in a contracted configuration.

FIG. 19 is a transverse cross sectional view of the device shown in FIG. 18, taken along lines 19-19.

FIG. 20 is an elevational view of the device of the invention shown in FIG. 18, in an expanded configuration.

FIG. 21 is a transverse cross sectional view of the device shown in FIG. 19, taken along lines 21-21.

FIG. 22 is an elevational view of another embodiment of the occlusive device of the invention having a mesh member comprising bundled strands intermittently spaced in a plurality of sections of the tubular member.

FIG. 23 is an elevational view of another embodiment of the occlusive device of the invention having a mesh member comprising woven strands disposed at the first end of the tubular member.

FIG. 24 is a transverse view of the mesh member, shown in FIG. 23, comprising woven strands.

FIG. 25 is a longitudinal cross sectional view of the device shown in FIG. 23, epithelialized in a body lumen.

FIG. 26A is a transverse cross sectional view of the device shown in FIG. 25, taken along lines 26-26.

FIG. 26B is a perspective view of another embodiment of this invention wherein the two different metals are sheets of metal coiled around each other.

FIG. 26C is a perspective view depicting another embodiment with the galvanic action formed by granules of different types of metal scattered along the outer surface of a cylindrical occlusive device.

FIG. 27 is a side view partially in cross-section illustrating another embodiment of the occlusive device having a mesh layer on an outer surface of the tubular member, within a body lumen.

FIG. 28 is a side view partially in cross-section of the tubular body of FIG. 27 in an expanded configuration.

FIG. 29 is a side view of a stent like tubular body with a metal rod within the tubular body.

FIG. 30 is a side view of the tubular body of FIG. 29 in an expanded configuration with metal mesh therein.

FIG. 31 is a side view of the occlusive device of the invention wherein the tubular element is comprised of two helical coils with their coils interlaced.

FIG. 32 is a side view of the occlusive device of the invention wherein the tubular element is comprised of two helical coils, one within the other.

FIG. 33 is a cross-sectional drawing of an occlusive device of the invention having a metal wire, metal mesh and stent-like structure.

FIG. 34A is a side view partially in cross-section with the delivery catheter with tissue abrading tip shown in a body lumen.

FIG. 34B is a side view partially in cross-section of the delivery catheter of FIG. 34A after the expulsion of the occlusive device into the body lumen.

FIG. 35A is a side view partially in cross-section of another embodiment of the invention as shown in FIG. 34A wherein the tissue-abrading portion is retractable.

FIG. 35B is a side view partially in cross-section of the embodiment shown in FIG. 35A with the tissue-abrading portion of the catheter shown in its expanded configuration.

FIG. 35C is a side view partially in cross-section of the embodiment shown in FIGS. 35A and 35B after the delivery of the occlusive device and retraction of the abrading portion.

FIG. 36A is a side view partially in cross-section of an embodiment of the delivery catheter after initial deployment.

FIG. 36B is a side view of the handle of the delivery catheter of the invention prior to deployment.

FIG. 37 is a side view cross-section of the handle of the deployment catheter.

FIG. 38 is a cross sectional view of the handle of FIG. 37 taken along the line 38-38 in FIG. 37.

FIG. 39A is a side view, partially in cut-away, of a distal tip of a delivery catheter having a closeable distal end.

FIG. 39B is a side view, partially in cut-away, of the distal tip of the delivery catheter of FIG. 39A showing the occlusive device being expelled from the distal tip of the catheter.

FIG. 39C is a side view, partially in cut-away, of the distal tip of the delivery catheter of FIG. 39B after the complete delivery of the occlusive device.

FIG. 40 is a longitudinal cross section view of the distal tip portion of the catheter in FIGS. 39A through 39C.

FIG. 41A is a side view partially in cross-section of another embodiment of the invention prior to delivery of the occlusive device.

FIG. 41B is a side view partially in cross-section of another embodiment of the invention during delivery of the occlusive device.

FIG. 41C is a side view partially in cross-section of another embodiment of the invention after the delivery of the occlusive device.

FIG. 42 is a transverse cross sectional view of the device shown in FIG. 41A, taken along lines 42-42.

FIG. 43A is a longitudinal cross section view of an uninflated balloon catheter for use as the delivery device of the present invention.

FIG. 43B is a longitudinal cross section view of an inflated balloon catheter for use as the delivery device of the present invention.

FIG. 43C is a longitudinal cross section view of another aspect of the inflated balloon catheter for use as the delivery device of the present invention.

FIG. 44A is a longitudinal cross section view of the occlusive device of the present invention including a self-expanding stent in a collapsed state.

FIG. 44B is a longitudinal cross section view of the occlusive device of the present invention including a self-expanding stent in an expanded state.

FIG. 45 is a side view of the balloon used in the balloon catheter for delivery of the device of the present invention.

FIG. 46 is a side perspective view of one embodiment of the occlusive device of the present invention.

FIG. 47 is a side perspective of another embodiment of the occlusive device of the present invention.

FIG. 48A is a side view of the occlusive device shown in FIGS. 46 and 47 encased in a stent-like structure.

FIG. 48B is another embodiment of the occlusive device encased in a stent-like structure.

FIG. 49 is a side cross sectional view of a disc used as an immediate barrier for use with the occlusive device of the present invention.

FIG. 50 is a transverse cross sectional view of the disc shown in FIG. 49, taken along lines 50-50.

FIG. 51 is a side view of the occlusive device of the present invention having an impermeable membrane stretched over a stent-like structure that encases the device.

FIG. 52A is a side view partially in cross section of the occlusive device in a collapsed state with a membrane stretched in umbrella-like fashion on the device.

FIG. 52B is a side view of the occlusive device in an expanded state with a membrane stretched in umbrella-like fashion on the device.

FIG. 53 is a side view partially in cross section of a delivery catheter having a bulbous impermeable end as an occlusive device.

FIG. 54 is a side elevational view of the bulbous impermeable end implanted in a vas of a male patient.

FIG. 55 is a side view partially in cross section of the bulbous impermeable end implanted in a body lumen.

### DESCRIPTION OF THE INVENTION

As used herein, tissue ingrowth includes but is not limited to cell multiplication and/or growth resulting in tissue formation into, onto, or surrounding a particular region and/or into, onto or surrounding an occlusive device. This may be epithelization, scar formation, or other cell growth or multiplication.

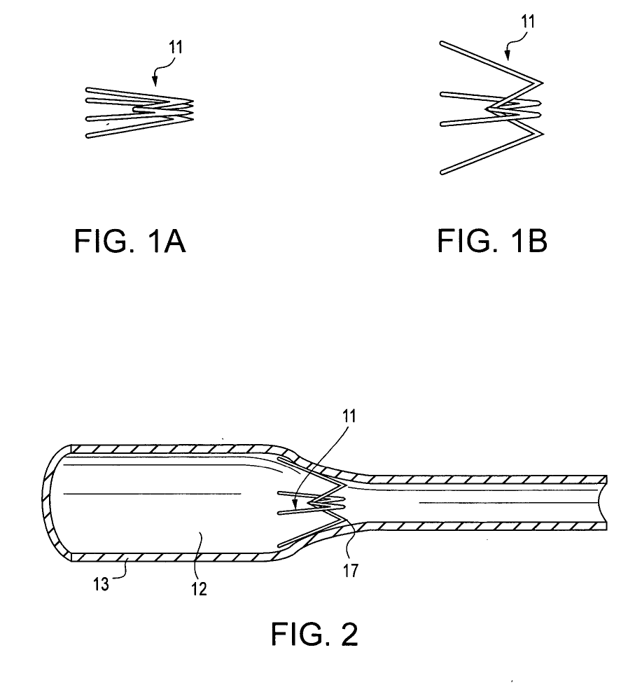

FIGS. 1A and 1B depict an occlusive device 11 of the present invention. In accordance with one aspect of this invention, the occlusive device 11 is delivered through a suitable delivery cannula (e.g., a tube or as a micro catheter or hypotube). Referring to FIG. 1A, the occlusive device 11 is constrained in a collapsed state while in a delivery device 10. Referring now to FIG. 1B, upon exiting the delivery device 10, the occlusive device 11 expands to an expanded state by the release of a radially expansive force.

Referring to FIG. 2, the configuration for the device 11 includes a central point from which individual struts or legs on an expandable frame 17 radiate outward. The expandable frame 17 of the device 11 exerts a constant outward force on the walls 13 of the fallopian tube or other passageway to maintain its position within a lumen 12 of the body. Sometimes at least one leg of the expandable frame 17 may be formed of thin, relatively rigid material that will lodge in the lumen wall 13 to secure the device 11 in place. The expandable frame 17 may even be in the form of a hook at the end to firmly secure the device 11 in place by the lumen wall 13.

It will be appreciated that, although a collapsible/expandable occlusive device 11 comprising a single unit is shown in FIGS. 1A, 1B, and 2, the device 11 may comprise a number of such units aligned adjacent to each other to form a length of an occlusive device 11 may even be joined together to form a single unit. Similarly, a tubular unit may be constructed so that it is expandable or collapsible as with a stent. Devices of this general nature are described in U.S. Pat. No. 6,096,052 to Callister et al. and U.S. Pat. No. 6,432,116 to Callister et al., the complete disclosures of which are incorporated herein as if set forth in full.

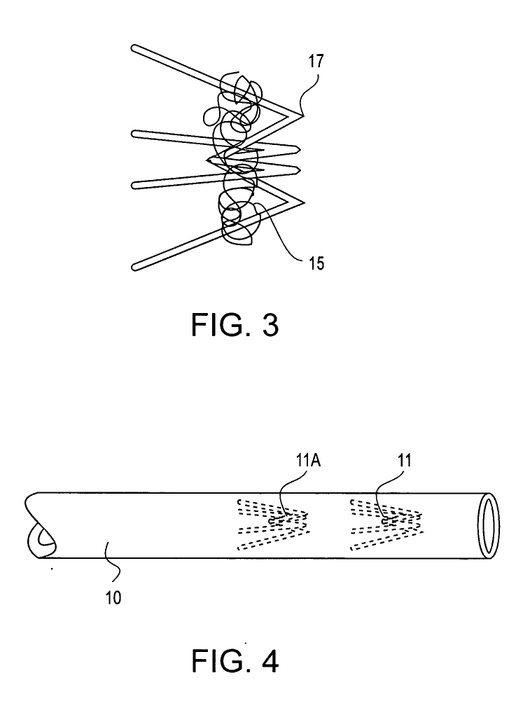

Referring to FIGS. 2 and 3, the occlusive device 11 may incorporate materials or mechanisms that may promote epithelialization within body tissues to create a more effective occlusion of the lumen 12 or result in a more secure attachment of the occlusive device 11 to the walls 13 of the body lumen 12. For instance, polyester fibers 15 may be attached to one or more expandable frames 17 of the device 11 such that tissue ingrowth into and around the device 11 form a plug, thereby occluding the passageway. A current may be passed into the body location where the device 11 is placed to enhance cell growth for attachment or cell ingrowth. The current may be provided by the device 11 by, for example, a galvanic current resulting from the materials used in the device 11.

Additionally, as described in detail below, substances such as therapeutic agents, drugs, (e.g., contraceptive hormones, spermicidal agents, spermatogenesis inhibitors, anti-microbials, antibiotics, anti-fungals, chemotherapeutic agents, etc.) or biological factors (VEGf, FGF, etc.) may be incorporated on or within the occlusive device 11 in order to bring about some desired effect (e.g., to accelerate tissue ingrowth, prevent/treat infection, cause drug-induced contraception for at least a sufficient period of time to allow the implanted lumen occlusive device to become fully functional, treat a disease or disorder in the adjacent tissue, etc). When the occlusive device 11 is used to block the lumen 12 of a fallopian tube, vas deferens or other body lumen for the purpose of deterring pregnancy, the lumen blocking efficacy of the device 11 (and thus its reliability as a contraceptive measure) may not become maximized for several weeks or months after the initial implantation of the device 11. Accordingly, a certain amount of time may be required for the occlusive device 11 to become fully epithelialized or for other tissue ingrowth to become complete. In such instances, a quantity of a contraceptive agent and/or spermicidal agent may be incorporated on or in the device 11 so as to provide for drug-induced contraception for a period of time that is at least sufficient to allow the lumen blocking efficacy of the device 11 to become maximized. The substance eluting implantable occlusive devices 11 of the present invention may be useable in various applications. For example, as described above, in applications where the device 11 is implanted in a fallopian tube or elsewhere in the female genitourinary tract for the purpose of blocking egg migration or implantation, the device 11 may additionally elute or deliver a female contraceptive agent or spermicidal agent to deter pregnancy, at least for some initial period of time following implantation of the intraluminal device. Any effective contraceptive or spermicidal agent may be used, in amounts that result in the desired therapeutic effect of avoiding pregnancy.

Specific examples of contraceptive agents that may be used include; the contraceptive hormone contained in the Norplant system (e.g., a synthetic progestin, namely, levonorgestrel having the molecular formula (d(−)-13-beta-ethyl-17-alpha-ethinyl-17-beta-hydroxygon-4-en-3-one) and a molecular weight of 312.45 and/or various other contraceptive hormone preparations including but not limited to medroxyprogesterone acetate, norethisterone enanthate, progestogen, levonorgestrel, levonorgestrel (as progestogen), ethinyl estradiol (as estrogen), norgestrel (as progestogen), levonorgestrel in combination with ethinyl estradiol, Norethisterone enanthate, norgestrel in combination with ethinyl estradiol, quinacrine, etc. Quinacrine is not a hormone. Rather, quinacrine is an agent which may be used to cause chemical, non-surgical female sterilization. When a quinacrine hydrocholoride pellet is inserted directly into the uterus, the guinacrine liquefies and flows into the fallopian tubes, causing permanent scarring. Although recorded failure rates and persistent side effects related to quinacrine sterilization have been low, controversy has developed around quinacrine's long-term safety, efficacy, and link to upper genital tract infections. However, direct placement of quinacrine into the fallopian tube in combination with or as part of a lumen blocking implantable device of this invention may permit the use or relatively low levels of quinacrine which would facilitate a local effect within the fallopian tube without untoward systemic toxicity.

Specific examples of specific spermicidal agents that may be used include but are not limited to nonoxynol-9, octoxynol-9, menfegol, benzalkonium chloride and N-docasanol.

Also, in any application where infection or microbial infestation is a concern, the device 11 may elute or deliver antimicrobial agent(s) (e.g., microbicidal agents, antibiotics, antiviral agent(s), anti paracyte agent(s), etc.) Specific examples of antimicrobial agents that may be eluted or delivered from the occlusive device 11 include but are not limited to: Acyclovir (Zovirax®); Amantadine (Symmetrel®); Aminoglycosides (e.g., Amikacin, Gentamicin and Tobramycin); Amoxicillin; Amoxicillin/Clavulanate (Augmentin®); Amphotericin B (Fungizone®); Ampicillin; Ampicillin/sulbactam (Unasyn®); Atovaquone (Mepron®); Azithromycin (Zithromax®); Cefazolin; Cefepime (Maxipime®); Cefotaxime (Claforan®); Cefotetan* (Cefotan®); Cefpodoxime (Vantin®); Ceftazidime; Ceftizoxime (Cefizox®); Ceftriaxone (Rocephin®)); Cefuroxime* (Zinacef®); Cephalexin (generic); Chloramphenicol; Clotrimazole (Mycelex®)); Ciprofloxacin (Cipro®); Clarithromycin (Biaxin®); Clindamycin (Cleocin®)); Dapsone; Dicloxacillin; Doxycycline; Erythromycin; Fluconazole (Diflucan®); Foscarnet* (Foscavir®); Ganciclovir* (Cytovene®) DHPG); Gatifloxacin* (Tequin®)); Imipenem/Cilastatin* (Primaxin®); Isoniazid* (generic), Itraconazole+ (Sporanox®); Ketoconazole; Metronidazole; Nafcillin; Nafcillin; Nystatin (generic); Penicillin; Penicillin G; Pentamidine; Piperacillin/Tazobactam (Zosyn®)); Rifampin (Rifadin®); Quinupristin-Dalfopristin (Synercid®); Ticarcillin/clavulanate* (Timentin®); Trimethoprim/Sulfamethoxazole (Bactrim®); Valacyclovir (Valtrex®); Vancomycin; Mafenide (Sulfamylon®); Silver Sulfadiazine (Silvadene®); Mupirocin (Bactroban®); Nystatin (Mycostatin®); Triamcinolone/Nystatin (Mycolog II, Loprox®); Clotrimazole/Betamethasone (Lotrisone); Clotrimazole (Lotrimin); Ketoconazole (Nizoral®); Butoconazole (Femstat®); Miconazole (Monostat®); Tioconazole (Vagistat®), detergent-like chemicals that disrupt or disable microbes (e.g., nonoxynol-9, octoxynol-9, benzalkonium chloride, menfegol, and N-docasanol); chemicals that block microbial attachment to target cells and/or inhibits entry of infectious pathogens (e.g., sulphated and sulponated polymers such as PC-515 (carrageenan), Pro-2000, and Dextrin 2 Sulphate); antiretroviral agents (e.g., PMPA gel) that prevent HIV or other retroviruses from replicating in the cells; genetically engineered or naturally occurring antibodies that combat pathogens such as anti-viral antibodies genetically engineered from plants known as “Plantibodies,” agents which change the condition of the tissue to make it hostile to the pathogen (such as substances which alter vaginal pH (e.g., Buffer Gel and Acidform) or bacteria which cause the production of hydrogen peroxide within the vagina (e.g., lactobacillus).

In accordance with another aspect of this invention, there is provided a system wherein two or more occlusive devices 11 may be loaded into the delivery device 10 prior to delivery as depicted in FIG. 4.

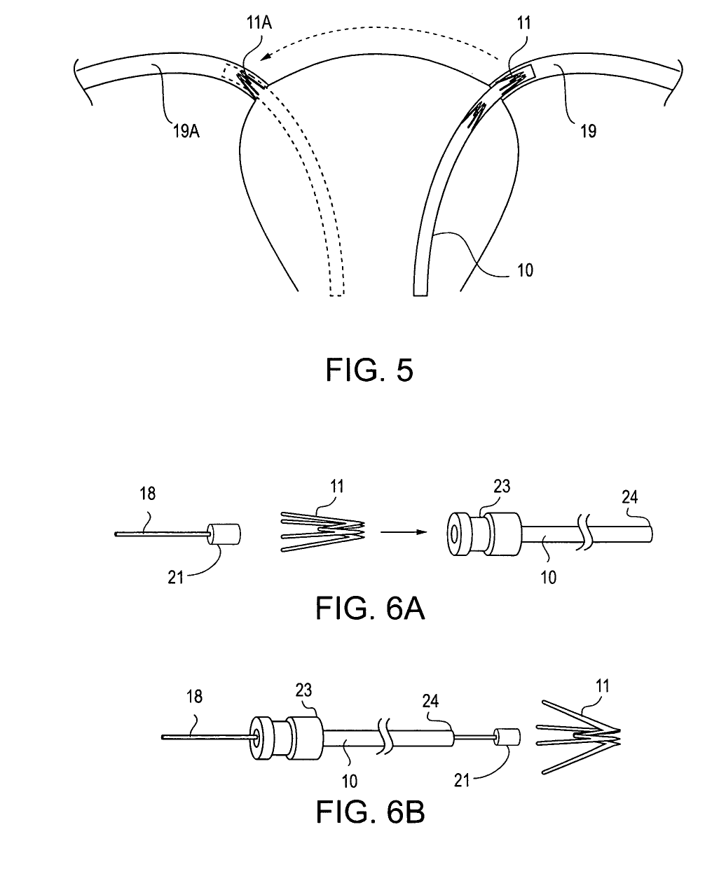

In the system shown in FIG. 4, if more than one device 11, 11A is to be delivered within the body, there is no need to remove the delivery device 10 to deliver the additional devices. For instance, if the device 11 being delivered is a fallopian tube occlusive device, the delivery device 10 may enclose two devices 11, 11A, one for each fallopian tube as shown in FIG. 5. In such an instance, the physician may insert the delivery device 10 into the uterus of the patient, and deliver one device 11 to a first fallopian tube 19, and, after delivery of the first device 11, he then could access a second fallopian tube 19A to place the second device 11A without having to withdraw the delivery device 10 from the uterus, he could merely navigate the delivery device 10 within the uterus without the need to withdraw it and reload an occlusive device. This has the advantage of speeding the overall procedure time since there is no need to remove and replace a delivery device 10 for each fallopian tube 19, 19A. Additionally, overall costs for the procedure are reduced since only one delivery device 10 is used to place two occlusive devices 11, 11A, rather than using two individual delivery devices 10.

Referring to FIGS. 6A and 6B, the present invention also allows for the occlusive device 11 to be advanced through the entire length of the delivery device 10. In such an instance, the delivery device 10 is advanced to the location where the occlusive device 11 is to be placed. A separate removable guide wire 18 may aid in positioning the delivery device 10. Following acceptable placement of the delivery device 10, the guide wire 18 is then removed from the delivery device 10 and the first occlusive device 11 is threaded into the proximal end 23 of the delivery device 10. After the occlusive device 11 is located within the proximal end 23 of the delivery device 10, it is then advanced through the length of the delivery device 10 using a pusher 21 that releasably engages the occlusive device 11 in order to push it through the delivery device 10.

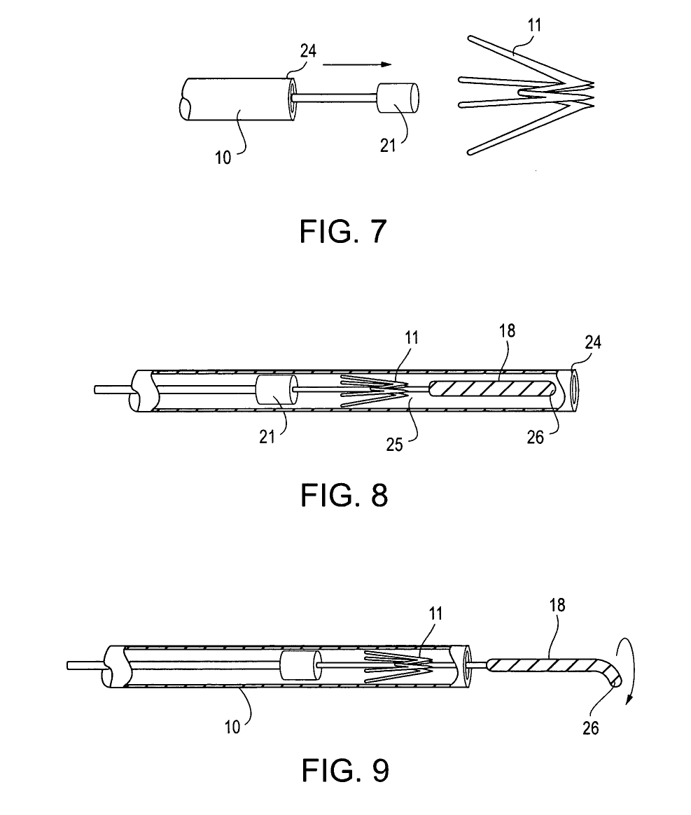

Referring now to FIG. 7, the occlusive device 11 is then advanced to the distal end 24 of the delivery device 10 where it is delivered in the desired location by pushing the device 11 out the distal end 24 of the delivery device 10.

In accordance with yet another aspect of this invention, there is provided a system wherein the occlusive device 11 is loaded or positioned within an annular compartment 25 between the guide wire 18 and the inner lumen of the delivery device 10, as shown in FIG. 8 below.

Located just proximal to the occlusive device 11 on the guide wire 18 is a section of guide wire 18 or similar structure, such as a pusher 21 that incorporates a larger diameter than the collapsed diameter of the occlusive device 11. The pusher 21 acts as interference with the occlusive device 11 so that as the guide wire 18 is advanced further distal through the delivery device 10, the occlusive device 11 is pushed along with it.

One major advantage to the type of system shown in FIG. 9 is that the entire system is steerable, since the distal portion 26 of the guide wire 18 may be torqued or steered through the body passageways to its desired location. Providing torque on the guide wire 18 has no significant effect on the occlusive device 11 since even in its collapsed state within the delivery device 10, there is still a small lumen through the occlusive device 11 through which the core of the guide wire 18 passes.

The flexible distal portion 26 of the guide wire 18 may incorporate a conventional spring tip or, alternatively, it may be made of or incorporate a plastic or Teflon coating to prevent any snagging of any attached fibers on the occlusive device 11. Additionally, the reduced diameter segment on the guide wire 18 where the occlusive device 11 rests may be longer than the occlusive device 11.

In an over-the-wire design there is a thin-walled hypo tube that pushes the occlusive device 11 out the end of the delivery device 10. A separate slideable guide wire 18 can then be advanced through the occlusive device 11. In this manner, a guide wire 18 may be placed in the fallopian tube first, and if un-navigateable or blocked, no occlusive device 11 is wasted. If the guide wire 18 can navigate into the tube, then the system can be used much like an over-the-wire stent placement, where the delivery device 10 and occlusive device 11 are then slid over the guide wire 18 and into the fallopian tube.

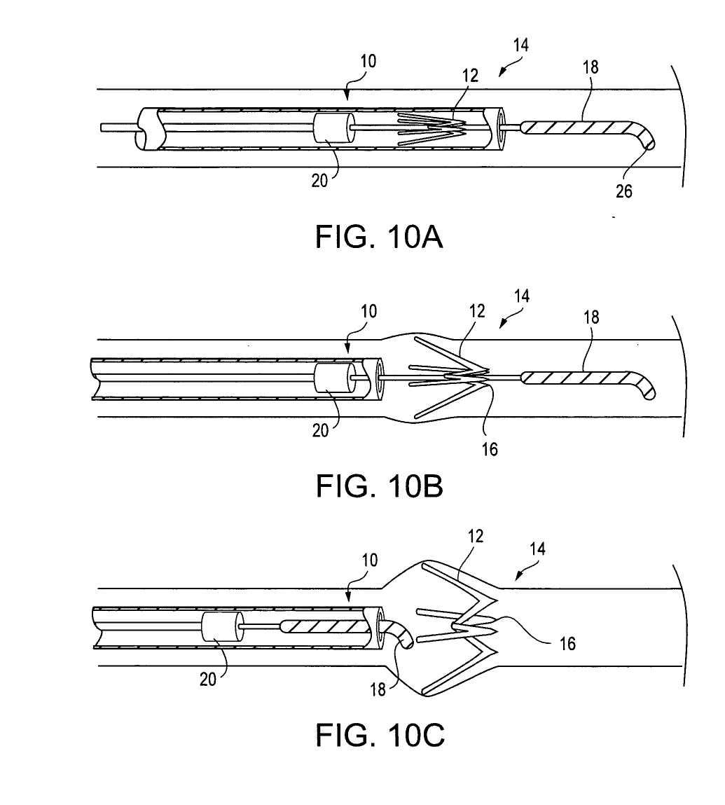

Referring now to FIG. 10A, a limited amount of axial movement of the guide wire 18, either proximally or distally, can be permitted to further aid in the steerability of the system. The delivery device 10 is thus able to provide either more or less support for the guide wire support, depending on the circumstances and the tortuosity of the vasculature or passageway being navigated.

As can be seen with reference to FIGS. 10B and 10C, once the delivery device 10 is steered to the desired location in the lumen 12, the occlusive device 11 is pushed out of the catheter lumen 12 and expands from its compressed configuration as shown in FIG. 10A into its expanded configuration as shown in FIGS. 10B and 10C. It may assume its expanded configuration as a result of temperature shape memory or release of compression, or any other appropriate means. Once it assumes its expanded configuration as shown in FIGS. 10B and 10C, it expands across the lumen 12 and assumes a configuration that has a large enough central opening 16 that the steerable end of the guide wire 18 can be retracted through the opening 16 and back into the lumen 12 of the delivery device 10 for withdrawal, leaving the occlusive device 11 in place in the lumen 12.

It can be appreciated that this system 14 may also be used to delivery very small diameter stents through the coronary or neuro-vasculature to areas of stenosis. In such a manner the moveable, but not removable, guide wire 18 gives the system 14 the advantage of a steerable guide wire 18 in combination with a very low profile delivery device 10.

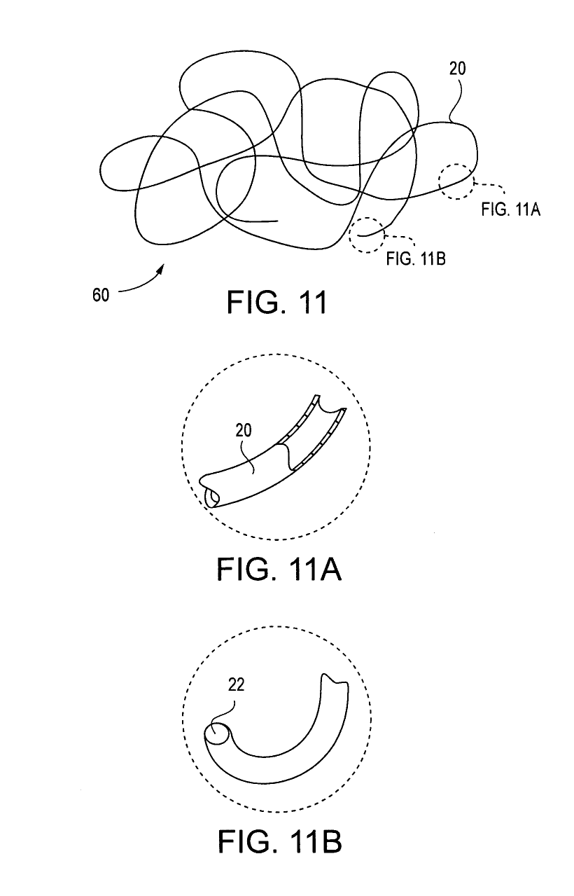

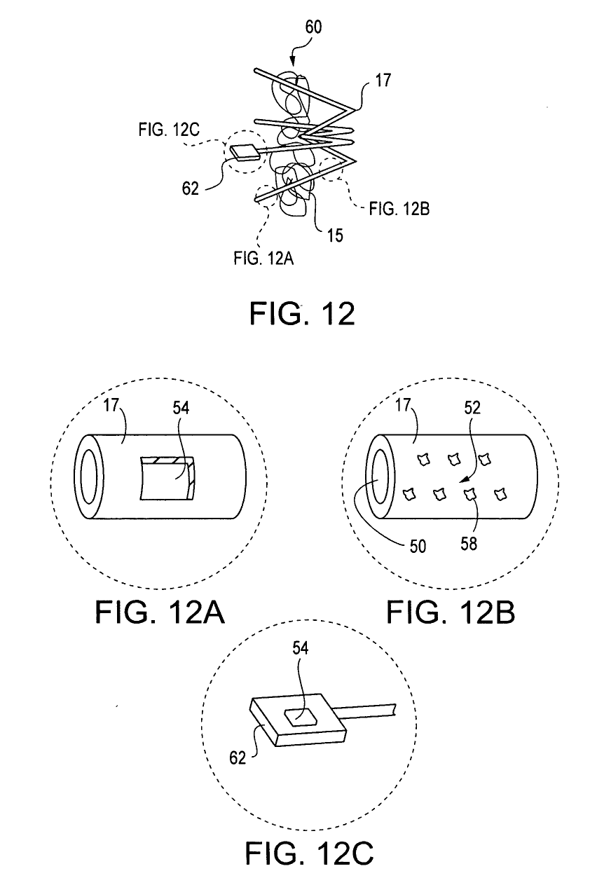

Referring now to FIGS. 11 and 12, there are provided implantable occlusive devices 11 that deliver or elute substance(s) (e.g., drugs, therapeutic agents, spermicides, biological factors, cell preparations, friendly microbes, etc.) for some period of time following implantation into the body. The substance-delivering implantable, intraluminal devices 60 may be of the configuration and structure of the occlusive device 11 previously described, may be configured as a drug eluting substance such as fibers 20 contained in a tubular structure, or may be of any other suitable configuration or structure. The rate and/or amount of substance delivered from the implanted device 60 may be designed or controlled, in accordance with known drug delivery technology, to both control dosage (e.g. concentration in the uterus, fallopian tube, lung, tumor or other tissue, organ or anatomical structure), the location of delivery (e.g. systemic, local, topical, directed downstream in a feeding artery, etc.) and the time period over which the drug or other substance would be eluded or otherwise delivered by the implanted device. Also, in some aspects, the delivery of a drug, therapeutic agent and the like may be responsive to the physical condition of the patient in question, such as drugs that are secreted into the uterus in response to the conditions presented at different times in the menstrual cycle, or different blood chemistry conditions during the diurnal cycle, or different conditions as a result of physical or medical conditions such as the presence of certain biological factors, the blood pressure presented, the blood flow encountered, or the like.

The substance that is to be eluted or delivered from the implanted intraluminal device 60 may be placed on or in the device 60 in various ways. For example, the device 60 may incorporate a mesh, tissue supporting member, lumen occluding member or other portion that is comprised of hollow fiber(s) 20 loaded with a drug or other substance, as shown in FIG. 11A.

Referring to FIG. 11B, each hollow fiber 20 could be extruded such that its inner diameter and/or outlet opening size 22 control the rate at which the drug or other substance will be eluted from or delivered by the device 60. The depth that the drug is loaded into the inner diameter 22 of each hollow fiber 20 would control the dispersment of the drug over time (i.e. more drug in the hollow fiber 20 will provide for a longer period of time over which the drug will be delivered).

As shown in FIG. 12 attached, the drug or substance may be enclosed within a semi-permeable reservoir 50 such that the drug or substance will diffuse or leach out of the reservoir 50 following implantation of the device 60 in a controlled manner based on the permeability of the reservoir 50 to the substance in question. The reservoir 50 may have a semi-permeable surface 52 such as a ceramic cylinder or the like, or may have a semi-permeable window 54 in an otherwise non-permeable container 62.

Referring now to FIG. 12C, the container 62 having the reservoir 50 may be attached to the device 60. The reservoir 50 may or may not be removable from the implanted device 60 and, in some embodiments, replaceable by another full reservoir 50 in situ while the device 60 remains in place. For example, in applications where the device 60 is implanted within a fallopian tube for the purpose of contraception, the reservoir 50 may be removed and/or replaced at a later date via a hysteroscope and a suitable removal device such as a gripping device or forceps passable through a working lumen of the scope. Alternatively, the reservoir 50 may be refillable, for example by a syringe.

The drug or substance may be mixed in to a material (e.g., a plastic) that oozes or otherwise passes out of the device 60 following implantation. In such embodiments, the molecules of the drug or substance may be sized so as to migrate or pass between polymer chains of the plastic such that the drug or substance will leach or pass out of the plastic over a desired time period.

The drug or substance may make up or be incorporated into a coating that is extruded or applied over all or a portion of the material located in or on the device 60, such that the drug or substance will elute or pass out of the coating at a desired rate or over a desired time period.

Referring to FIGS. 12A and 12B, the drug or substance may make up or may be incorporated in a coating that is applied to all or a portion of the expandable frame 17 of the device 60 (e.g., a drug delivery coating on a self expanding nitinol or other metal frame) such that the drug or substance will elute or pass out of that coating at a desired rate or over a desired time period.

Referring to FIG. 12B. one or more holes 58 or indentations or other texture may be drilled or otherwise formed in the expandable frame 17 of the device 60 or other portion of the device 60 or in the fibers 15 and the desired drug or substance may be placed in the hole(s) 58 such that the drug or substance will elute or pass out of the hole(s) 58 over a desired time period. The diameter(s) and/or depth(s) of the hole(s) 58 may be selected to control the rate and time over which the drug or substance will elute or otherwise pass from the device.

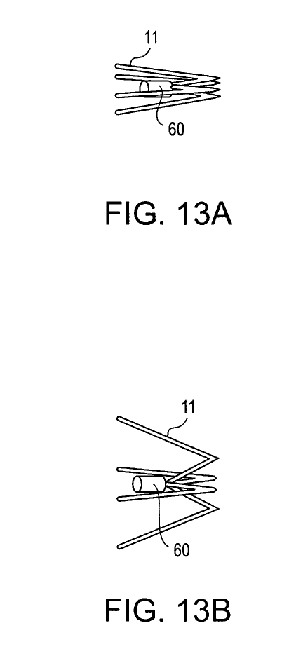

Referring to FIG. 13, a substance delivering implant 60 may be implanted separately from the occlusive device 11. For example, in embodiments where an occlusive device 11 is implanted in a fallopian tube for contraceptive purposes, a substance delivering implantable intraluminal device 60 may be placed in the lumen of the fallopian tube either proximally to, within, or distally to the occlusive device 11. The matrix of the substance delivering implantable intraluminal device 60, in some embodiments, may be biodegradable such that after a desired or predetermined period of time, the substance delivering implantable intraluminal device 60 would dissolve and be gone.

The substance may be responsive to the physiological conditions and thereby control the delivery of the substance in response to those conditions. For example, where the substance is released for contraceptive purposes within the fallopian tubes, the release of the substance may be controlled to some extent by the menstrual cycle of the patient. Certain well-known biochemical conditions prevail within the uterus and fallopian tubes at the time and shortly after the release of the egg from the ovaries (referred to here as ovulation). The device 60 with a spermicidal substance or other similar contraceptive substance may be coated with a substance that is soluble in response to the biochemical conditions that prevail at the time of ovulation, but relatively insoluble in the biochemical conditions that prevail in the uterus and fallopian tubes at other times. This would result in the release of the substance primarily at the time of ovulation, and thus result in a long lasting substance delivering implantable intraluminal device 60 that enhances contraception at precisely the time when it will be effective. Another example of the release of the substance in response to physiological conditions would be where a greater amount of substance is released in response to increased blood flow, as in a chemotherapeutic agent located in a feeding artery to a tumor. As the blood flow decreases, smaller amounts of the chemotherapeutic substance is released, resulting in decreased systemic effects as the blood flow to the tumor is cut off. Responses to blood pressure, diurnal cycles, and the like can also be engineered in accordance with this invention.

Another aspect of this invention provides for a means of placing the occlusive device 11 at the proper depth within a fallopian tube. In one embodiment, the distal end 24 of the delivery device 10 is colored a different color than the body of the delivery device 10. As the occlusive device 11 is advanced through a hysteroscope, the change in color on the distal end 24 of the delivery device 10 can be viewed through the hysteroscope as the distal end 24 of the delivery device 10 enters the fallopian tube.

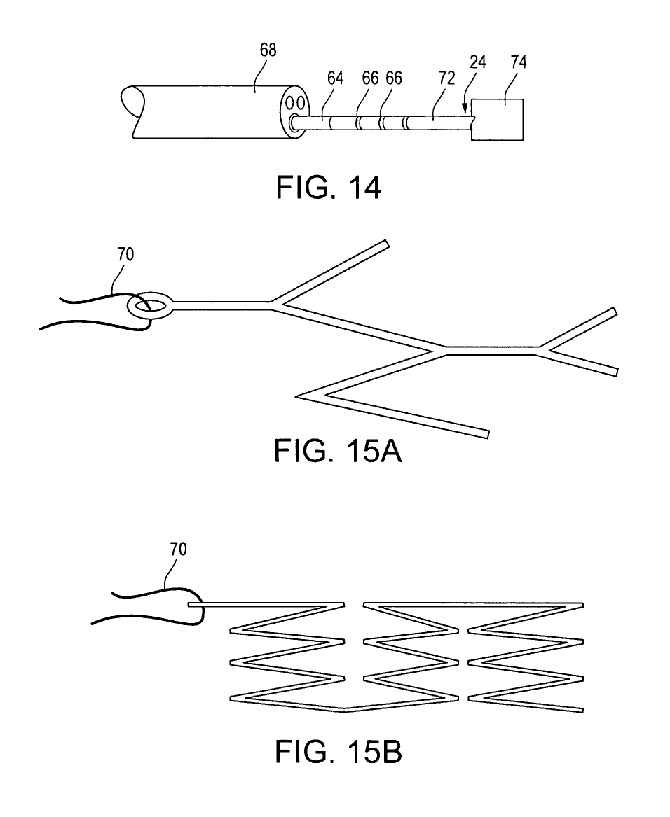

When the color changes and is completely located within the fallopian tube, the enclosed occlusive device 11 is properly located at the specified depth. The occlusive device 11 may then be delivered, ensuring that it is placed at a pre-specified depth within the fallopian tube. Depending on the length of the visual marker on the distal end 24 of the delivery device 10, the occlusive device 11 may be located within the isthmic region of the fallopian tube, distal to the isthmic region, or even near the ampulla region of the fallopian tube. Referring to FIG. 14, an alternative to the variable colored distal end 24 is a visual marker 64 on the delivery device 10. As the visual marker 64 enters the fallopian tube, the occlusive device 11 is at the proper depth for deployment. Alternatively, two visual markers 64, 66 may be placed to show a pre-specified range of depth indication proper placement. The visual markers 64, 66 may have any suitable form, for example a marker ring at the appropriate location along the length of the tube, or ruler type striations that indicate depth of insertion, or other similar visual indicia. For example, visual markers 66 on catheter shaft 72, make it possible for the operator to determine, for example in millimeters, how deep the device 11 is advanced into the fallopian tube or other lumen. While this generally requires greater ability to visualize the details on the catheter shaft 72, it may be preferable if, for example, the precise depth of placement of the device 11 is not known before the operation or if that depth of placement varies between operators, or if some other reason the operator wants to know the exact depth of placement for the device 11.

An alternative to visual means of placement is the use of ultrasound guidance. In this case, a marker that is echogenic is placed on the tip of the delivery catheter and a second market locating the occlusive device 11 within the delivery device 10 allows for proper placement of the occlusive device 11 under ultrasonic guidance.

Another means of placement for the device is under fluoroscopic guidance. In this case, a radiopaque marker is located at the tip of the delivery device 10 and a second marker locates the occlusive device 11 within the delivery device 10. When the proper depth of the delivery device 10 within the fallopian tube has been seen under fluoroscopy, the occlusive device 11 is ready to be deployed. Additionally, the occlusive device 11 may be made radiopaque, either in part or in whole, allowing for direct visualization under fluoroscopy and easier placement.

In another embodiment of the present invention, a physical barrier is used on the catheter shaft 72 to prevent over-insertion of a mechanical deployable flare 74 on the tip of the catheter shaft 72. This idea is to deploy and help denude the epithelia layer on the fallopian tube thus enhancing a tissue response. The flare 74 could both be deployed when entering the tube and/or when deploying the occlusive device 11.

As shown in FIGS. 15A and 15B, the device 11 may further comprise a flag or marker 70 that unravels or extends out of the fallopian tube and into the uterus for visual confirmation to indicate which fallopian tube has a device 11 in it. Optionally, the flag 70 can contain a substance (e.g., contraceptive drug, antifungal, antibiotic, agent for treatment of STD such as pelvic inflammatory disease, spermicidal agent, etc.) as described above. Also, optionally, the flag 70 may be dissolvable or biodegradable and/or retrievable and removable at a later date, such through an endoscopes or hysteroscope as described above. In embodiments, where the flag 70 or any other component of the device 11 is removable from the body, that component may contain substance(s), such as copper, that are desirable for only short term implantation.

Also, in some applications, a substance eluting implantable device 60 may be placed in a body lumen (e.g., blood vessel, bronchus, hepatic duct, common bile duct, pancreatic duct, etc.) near a tumor and the device 60 may deliver one or more anti-tumor agents to treat the tumor. Specific examples of anti-tumor agents that may be used in this invention include but are not limited to: Alkylating agents or other agents which directly kill cancer cells by attacking their DNA (e.g., cyclophosphamide, isophosphamide), nitrosoureas or other agents which kill cancer cells by inhibiting changes necessary for cellular DNA repair (e.g., carmustine (BCNU) and lomustine (CCNU)), antimetabolites and other agents that block cancer cell growth by interfering with certain cell functions, usually DNA synthesis (e.g., 6 mercaptopurine and 5-fluorouracil (5FU), Antitumor antibiotics and other compounds that act by binding or intercalating DNA and preventing RNA synthesis (e.g., doxorubicin, daunorubicin, epirubicin, idarubicin, mitomycin-C and bleomycin) Plant (vinca) alkaloids and other anti-tumor agents derived from plants (e.g., vincristine and vinblastine), Steroid hormones, hormone inhibitors, hormone receptor antagonists and other agents which affect the growth of hormone-responsive cancers (e.g., tamoxifen, herceptin, aromatase ingibitors such as aminoglutethamide and formestane, triazole inhibitors such as letrozole and anastrazole, steroidal inhibitors such as exemestane), antiangiogenic proteins, small molecules, gene therapies and/or other agents that inhibit angiogenesis or vascularization of tumors (e.g., meth-1, meth-2, thalidomide (Thalomid), bevacizumab (Avastin), squalamine, endostatin, angiostatin, Angiozyme, AE-941 (Neovastat), CC-5013 (Revimid), medi-522 (Vitaxin), 2-methoxyestradiol (2ME2, Panzem), carboxyamidotriazole (CAl), combretastatin A4 prodrug (CA4P), SU6668, SU11248, BMS-275291, COL-3, EMD 121974, IMC-1C11, IM862, TNP-470, celecoxib (Celebrex), rofecoxib (Vioxx), interferon alpha, interleukin-12 (IL-12) or any of the compounds identified in Science Vol. 289, Pages 1197-1201 (Aug. 17, 2000)), biological response modifiers (e.g., interferon, bacillus calmette-guerin (BCG), monoclonal antibodies, interluken 2, granulocyte colony stimulating factor (GCSF), etc.), PGDF receptor antagonists, herceptin, asparaginase, busulphan, carboplatin, cisplatin, carmustine, cchlorambucil, cytarabine, dacarbazine, etoposide, flucarbazine, flurouracil, gemcitabine, hydroxyurea, ifosphamide, irinotecan, lomustine, melphalan, mercaptopurine, methotrexate, thioguanine, thiotepa, tomudex, topotecan, treosulfan, vinblastine, vincristine, mitoazitrone, oxaliplatin, procarbazine, streptocin, taxol, taxotere, analogs/congeners and derivatives of such compounds as well as other antitumor agents not listed here.

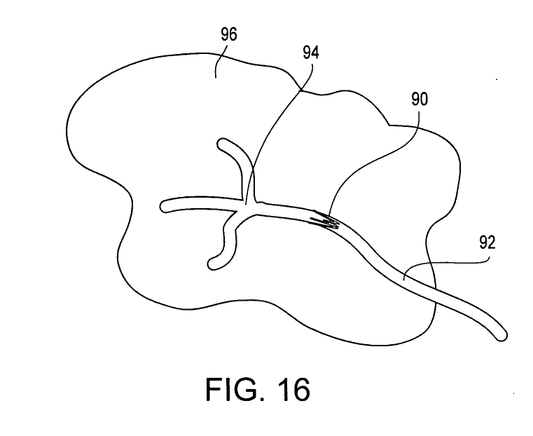

Referring to FIG. 16, in some embodiments used for antitumor applications, an intraluminal implant 90 may block or occlude the body lumen 94, for example a feeding artery 92 in which it is implanted. In other applications, the intraluminal device 90 may continue to allow some flow of body fluid through the body lumen 94 in which it is positioned and into the tumor 96. The intraluminal implant 90 may not immediately cause complete occlusion of the artery 92 such that some blood continues to flow into the tumor 96. The antitumor drug eluted by the intraluminal implant 90 is thus carried into the tumor 96 for some desired period of time following implantation. Thereafter, cellular ingrowth into the intraluminal implant 90 causes a progressive and complete occlusion of the artery 92 after the desired dose of antitumor drug has been delivered. This blockage of blood flow to the tumor 96 may further serve to inhibit or kill some or all of any remaining tumor cells that have not been killed by the antitumor drug. The release of the drug may be controlled based on the rate of blood flow through the feeding artery 92. As the artery 92 occludes over time, less total amount of the drug will be released into the bloodstream and thus there will be less systemic effects of the chemotherapeutic agent which will generally result in less dramatic side effects. On the other hand, the concentration of the chemotherapeutic agent will generally be slightly more concentrated in the blood based on the reduced flow, resulting in a more concentrated but more localized therapeutic effect on the tumor 96.

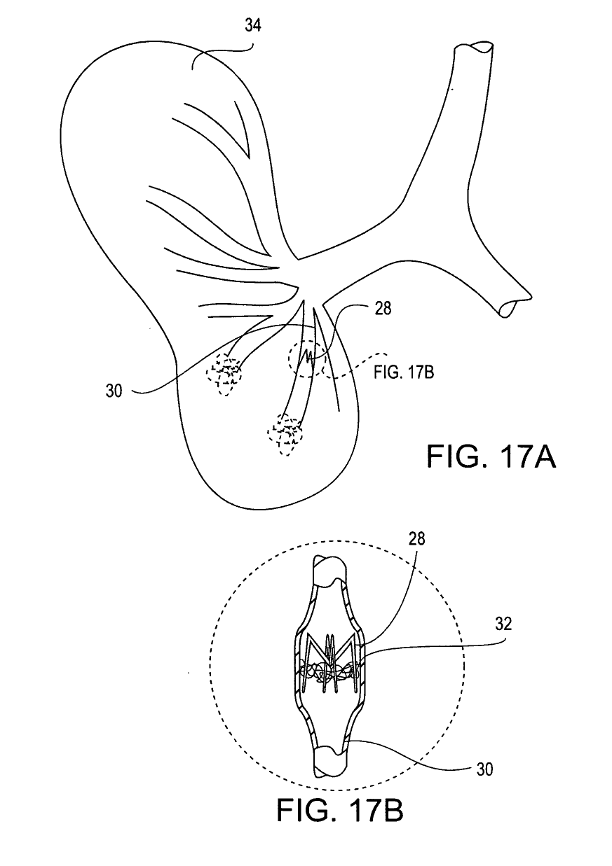

In yet another example of an application of this invention as shown in FIG. 17, the implantable intraluminal device 28 is implanted into a bronchus 30 of a lung 34. In some cases, this device 28 may cause instant or progressive full occlusion of the bronchus 30, so as to prevent air from entering a lobe or region of the lung 34 that is leaking or diseased, such as leakage that may occur due to a ruptured emphysematous bleb, traumatic lung puncture or iatrogenic lung rupture. In other cases the device 28 may be constructed so as not to substantially block airflow through the bronchus 30 and possibly even to perform a scaffolding or stenting function which holds the lumen of the bronchus 30 open. In either type of device 28, a drug or substance may be eluted or delivered by the device 28 into the adjacent pulmonary tissue. For example, in cases where the device 28 has been implanted to close off flow to a punctured area of the lung 34, the device 28 may elute an antibiotic or other agent either from the implantable intraluminal device 28 itself or from fibers 32 or other mesh contained in the device 28, to locally deter or treat any infection that may develop in the injured lung tissue. In cases where the device 28 is implanted in a bronchus 30 to treat emphysema or chronic obstructive pulmonary disease, the device 28 may elute a therapeutic agent that is effective to treat that underlying condition or its symptoms.

Some examples of drugs that may be eluted from the device for the purpose of treating such lung diseases include but are not limited to: antimicrobial substances (examples as listed above); corticosteroids such as beclomethasone (Vanceril, Beclovent), triamcinolone (Azmacort), flunisolide (Aerobid), fluticasone (Flovent), budesonide (Pulmicort), dexamethasone, prednisone, prednisolone, methylprednisolone (Medrol, SoluMedrol, DepoMedrol), methylprednisolone (Depo-Medrol), hydrocortisone (SoluCortef), methylprednisolone (SoluMedrol); Mediator-release inhibitors or cromones such as, cromolyn sodium (Intal), nedocromil sodium (Tilade); anti-leukotriene drugs such as leukotriene-receptor antagonists (e.g., zafirlukast (Accolate)), leukotriene-synthesis inhibitors (e.g., zileuton (Zyflo)) and other anti-leukotrienes (e.g., montelukast (Singulair)), mucolytic agents and expectorants (e.g., guifenisn); bronchodilator drugs such as beta-adrenergic agonists (e.g., epinephrine (Primatene), isoproterenol (Isuprel), isoetharine (Bronkosol), metaproterenol (Alupent, Metaprel), albuterol (Proventil, Ventolin), terbutaline (Bricanyl, Brethine), bitolterol (Tornalate), pirbuterol (Maxair), salmeterol (Serevent), Methyl xanthines (e.g., caffeine, theophylline, aminophylline and oxtriphylline (Choledyl)) and anticholinergics (e.g., atropine, ipratropium bromide (Atrovent).

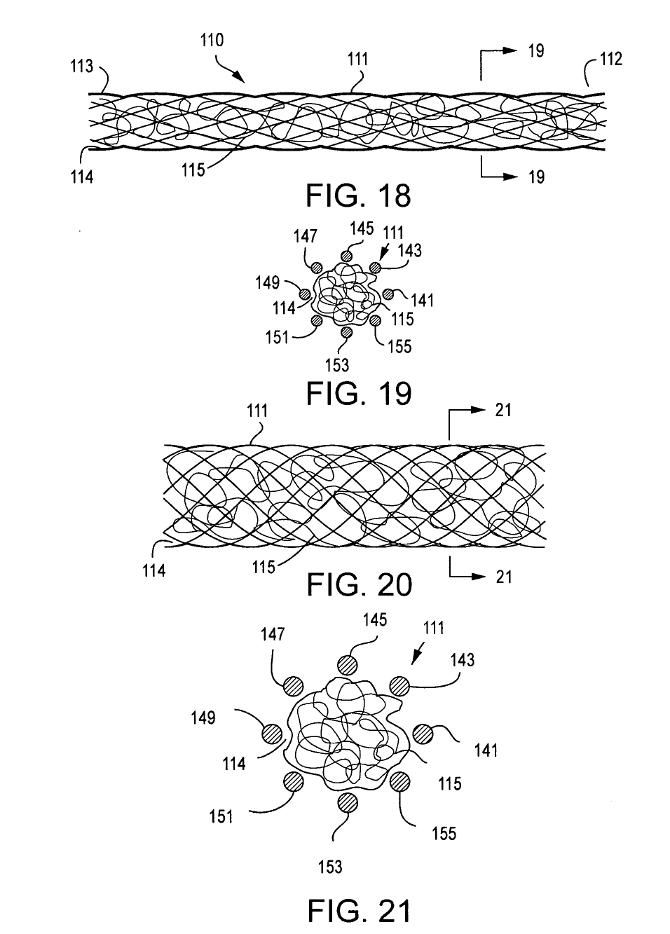

Referring to FIGS. 18-33, cell multiplication and ingrowth in general and specifically endothelial cell multiplication and ingrowth are generally desirable in this invention both for anchoring the occlusive device 110 and enhancing the occlusion created by the device 110. Cell multiplication and ingrowth in general and endothelial cell multiplication and ingrowth in particular may be enhanced by the application of a voltage to the cells or tissue location in question. For the applications of this invention, cell ingrowth is into the occlusive device 110, for example into a stent or single unit occlusive device. While fiber bundles or even single fibers may provide a framework onto which the cells may fix to form an occlusive device 110, and coatings or other similar substances or even the presence of the fiber itself my encourage or precipitate cell multiplication and/or growth of cells to create ingrowth, it has been found that the application of an electric potential to the area in question will also encourage and enhance ingrowth.

One method of applying an electric potential to the tissue is to make the occlusive device 110 of a conductive material and to attach a battery to the occlusive device 110. Another method would be to construct the occlusive device 110 of different metals and therefore form a galvanic battery out of the occlusive device 110. The device 110 would function as a galvanic battery using body tissue as the electrolyte, so that the device 110 would generally not be a galvanic battery in storage, but when implanted into the human body would function as a galvanic battery and thereby apply a voltage to the tissue surrounding the occlusive device 110, encouraging cell growth.

Still referring to FIGS. 18-33, the anode may be wire, ribbon, tubing, braid, mesh, fibers or other suitable configuration. The cathode may be a different metal and may be formed in the above configurations as well. Both materials may be interwoven with each other as in a screen or mesh, with some of the wires made of each material.

Alternatively, homogenous meshes or screens made of different metal from each other may be placed, in practice, in close proximity to one another to create an electrical potential between them. The use of mesh or screen may provide surface area for cell ingrowth and may simultaneously provide a framework for cell ingrowth.

The galvanic structure need not be a mesh. For example, another advantageous embodiment is having the two different metals in the form of a ribbon and being in a coiled configuration, where the ribbons are not in direct contact with each other. Likewise stents made of units made of alternating and insulated metal units or alternating and insulated wires will provide a similar advantage. One spring inside another or one stent inside another separated by an insulating fiber bundle would, likewise, create a galvanic battery while providing a structure for the ingrowth of tissue. Of course, for various purposes including controlling the voltage created at a particular location, there may be more than two different metals from which the ribbons are made, or may be more than two different ribbons. Depending on the metals for example, there may be a common anode and two different cathodes, or visa versa, creating different potentials at different locations.

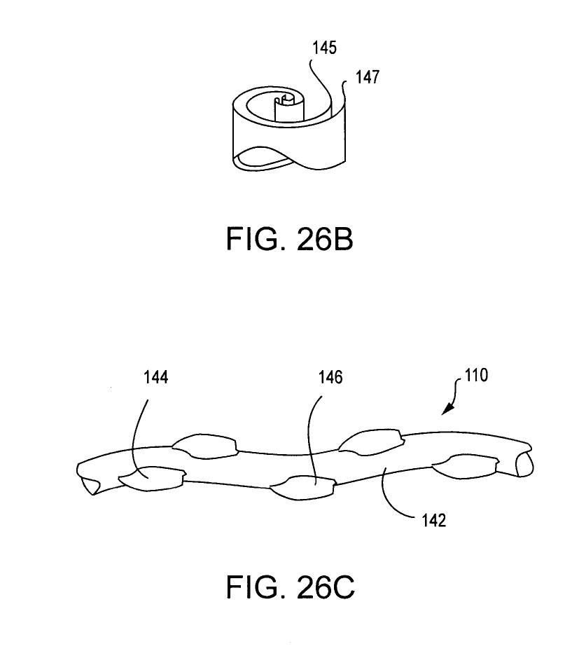

Alternatively, conductive plastics or metal impregnated plastics or fibers may be used to create the galvanic structure to create an electrical potential to encourage or enhance cell division or cell growth. For example, as shown in FIG. 26C, metal flakes or grains 144,146 may be embedded in or attached on a single fiber 142 and may create an electrical potential when placed in the body.

Referring specifically to FIG. 18 an occlusive device 110 embodying features of the present invention generally comprises a tubular member 111 having a first end 112, a second end 113, and a lumen 114 extending therein. As best shown in FIG. 19, illustrating a transverse cross section of the tubular member 111 shown in FIG. 18 taken along lines 19-19, a mesh member 115 is transversely disposed on the tubular member 111. In a presently preferred embodiment, the occlusive device 110 is a contraceptive or sterilization device for occluding a reproductive body lumen 121. In this embodiment, in order to generate the galvanic action, the tubular member 111 is formed of woven or interspersed wire elements 141, 143, 145, 147, 149, 151, 153 with the different wires made of at least two different metals. For example, wires 141, 145, 149 and 153 may be made of stainless steel, where 143, 147, 151 may be made of gold. While the galvanic effect generated by those two biocompatible metals might be very slight, the mild electrical irritation may be sufficient to stimulate the desired ingrowth.

Similarly, again with reference to FIGS. 18 and 19, the wire elements 141, 143, 145, 147, 149, 151, 153 may be one metal, for example stainless steel, and the mesh 115 may be another, for example copper wool. In this case, the copper wool not only acts to generate a galvanic response, but also may aid in the contraceptive effect of the occlusive device 110.

In the embodiment illustrated in FIGS. 18 and 19, the tubular member 111 is in its relatively small-dimensioned configuration for introduction and advancement into the patient's body lumen 121. FIG. 20 illustrates the tubular member 111 shown in FIG. 18 in an open, relatively large dimension configuration. As illustrated in FIG. 21, showing a transverse cross section of the tubular member 111 shown in FIG. 20 taken along lines 21-21, the mesh member 115 expands so that it extends across the expanded lumen 114 of the tubular member 111. In this configuration the tubular member 111 has an open, lattice-type structure facilitating epithelialization which secures the occlusive device 110 to the wall defining the body lumen 121. Preferably, tubular member 111 can be deformed to an expanded diameter, preferably equal to or slightly larger than the dimensions of the body lumen 121 within which the contraceptive device 110 is to be disposed. For disposition within a female patient's fallopian tubes the expanded transverse dimensions should be about 0.1 mm to about 5 mm.

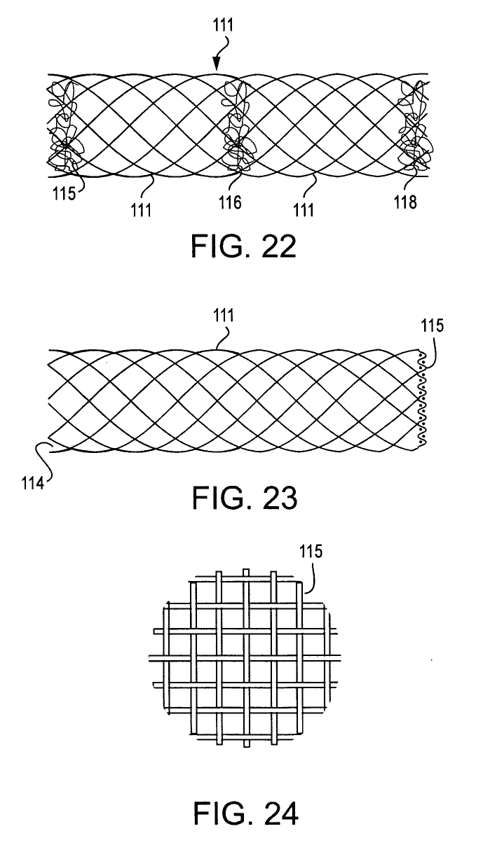

The mesh member 115 is permeable to allow for tissue ingrowth. The permeability of the mesh member 115 facilitates epithelialization, and the epithelialized mesh 115 occludes the reproductive body lumen 121 sufficiently to prevent the passage of reproductive cells therethrough. In a presently preferred embodiment, the mesh member 115 comprises intertwined strands of a biocompatible material connected to the tubular member 111. In the embodiment illustrated in FIG. 18, the mesh member 115 comprises bundled strands. In the embodiment illustrated in FIG. 23 the mesh member 115 comprises woven strands. FIG. 24 is a transverse view of the device illustrated in FIG. 23, illustrating the woven strands forming the mesh member 115. However, referring to FIG. 22, the mesh member 115, 116, 118 may comprise a variety of suitable permeable structures which support epithelialization, as for example, where the mesh member comprises the walls of the tubular member 111 connected together to form a closed end of the tubular member 115 (not shown). The biological response of tissue ingrowth is stimulated and enhanced by the galvanic action generated by the construction of the occlusive device 110 as described above.

In the embodiment illustrated in FIG. 18, the mesh member 115 extends along the length of the tubular member 111 from the first end 112 to the second end 113 thereof. In another embodiment, illustrated in FIG. 22, the mesh members 115, 116, 118 are disposed in a plurality of sections intermittently spaced along the length of the tubular member 111. In such a configuration, if the fiber bundles 115, 116, 118 are of different metal, and are separated from each other along the length of the tube 111o, and if the material from which the tubular member 111 is made is a conductor, a galvanic potential between the bundles of fiber may be generated.

FIG. 23 illustrates another embodiment, in which the mesh member 115 is disposed at the first end of the tubular member 111. In the embodiment illustrated in FIG. 23, the mesh member 115 comprises a single sheet of woven material, disposed in the lumen 114 of the tubular member 111. Alternatively, a plurality of stacked woven mesh sheets may be provided, including sheets having different mesh sizes. To create galvanic action in such situations, the wires forming the woof and warp of the woven material may be of different metals. Alternatively, a series of stacked woven mesh sheets are used, the mesh sheets may be made of different metals.

In the embodiments illustrated in FIGS. 18, 22, and 23, the mesh member 115 is within the lumen 114 of the tubular member 111. The mesh member 115 may be connected to the tubular member 111 by a variety of suitable means including adhesive, heat bonding, or solvent bonding.

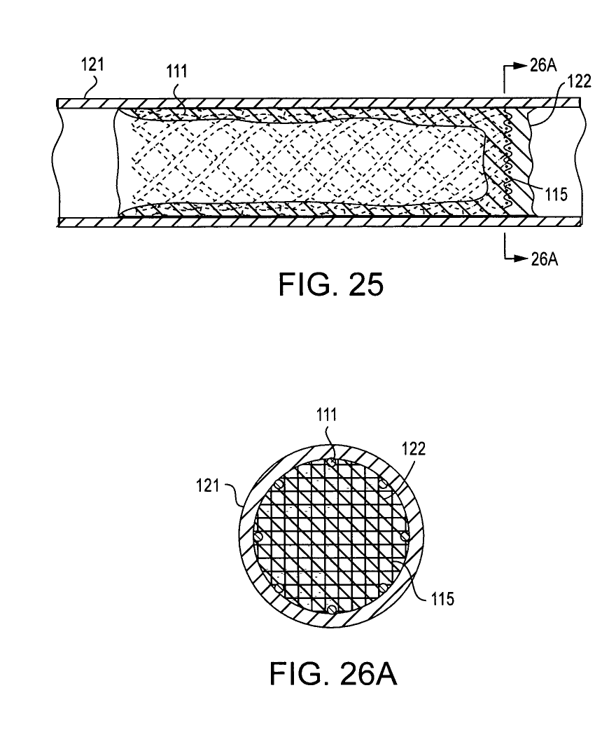

The tubular member 111, expanded within the body lumen to be occluded, epithelializes to secure the occlusive device 110 within the body lumen 121, and tissue ingrowth in the mesh member 115 occludes the lumen 114 of the tubular member 111 and the body lumen 121. FIG. 25 illustrates the embodiment of the occlusive device 110 shown in FIG. 23, installed within the patient's body lumen 121, with tissue ingrowth 122 within the walls of the tubular member 111 and within the mesh member 115. FIG. 26A illustrates a transverse cross section of the installed occlusive device 110 shown in FIG. 25 taken along lines 26A-26A.

FIG. 26B shows another embodiment of this invention wherein the two different metals are sheets of metal coiled around each other. FIG. 26C depicts yet another embodiment with the galvanic action formed by granules of different types of metal scattered along the outer surface of a cylindrical occlusive device 110.

A variety of materials may be used to form the mesh member 115, including plastics, polymers, metals, and treated animal tissues. In a presently preferred embodiment, the mesh member 115 is an irritant, such as Dacron or Nylon, which promotes epithelialization. Additionally, the mesh member 115 may be coated or otherwise impregnated with cell growth stimulators, hormones, and/or chemicals to enhance tissue impregnation. The fibers used to form the mesh member 115 are generally about 0.00025 mm to about 0.25 mm in diameter. It would be obvious that a wide variety of mesh sizes which support epithelialization may be used. For example, in one embodiment the mesh member 115 mesh size is about 5 mu.m to about 0.05 mm, and preferably about 10 mu.m to about 15 mu.m. Preferably, mesh members 115, 116, 118 having relatively large mesh sizes are coated with the epithelialization promoter agents.

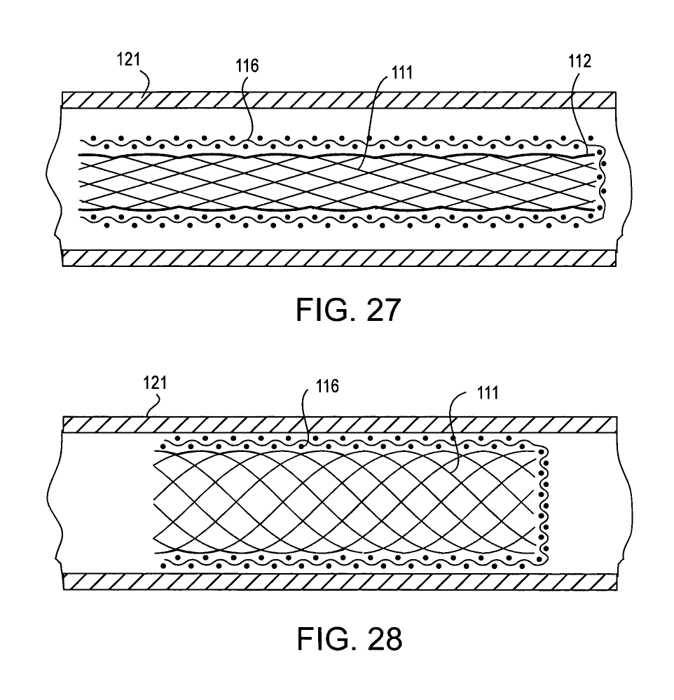

In one embodiment, illustrated in FIG. 27, a mesh layer 116 is provided along at least a section of the outer surface and/or the inner surface of the tubular member 111, to facilitate tissue epithelialization along the tubular member 111 and into the mesh member 115. In the embodiment illustrated in FIG. 27, the mesh layer 116 is disposed along the entire length of the outer surface of the tubular member 111 and transversely disposed at the first end 112 of the tubular member 111. The mesh layer 116 may be an integral extension of the mesh member 115, or a separate member connected to or separate from the mesh member 115. In a presently preferred embodiment, the mesh layer 116 comprises woven or bundled strands of a, preferably, biocompatible material, which may be a single or a plurality of mesh sheets, as discussed above in connection with the mesh member 115. The mesh layer 116 is permeable to allow for tissue ingrowth, and consequently, facilitates ingrowth within the mesh member 115, as for example, in embodiments in which only a section of the tubular member 111 is expanded into contact with a wall of the body lumen 121, as discussed below. In this embodiment, the galvanic action may be created by using different metals for the exterior mesh of the tubular member 111, or by constructing the tubular member 111 of wire elements of at least two different metals.

The tubular member 111 may be expanded in the body lumen 121 using a balloon catheter, or alternatively, it may be self-expanding. The tubular member 111 is preferably self expanding in the embodiment in which the mesh member 115 is disposed along the length of the tubular member 111, as in the embodiment illustrated in FIG. 18, or is disposed at least in part at the second end 113 of the tubular member 111, as in the embodiment illustrated in FIG. 22.

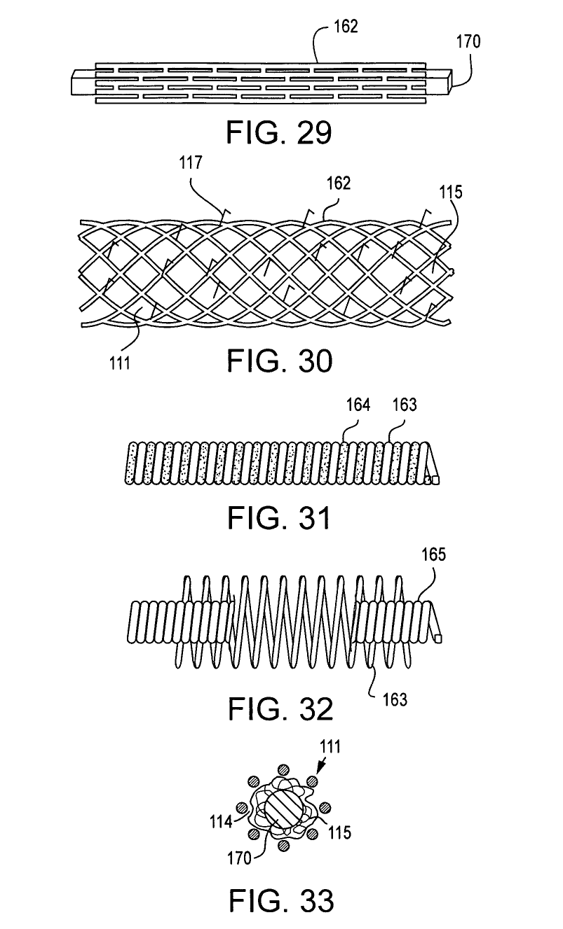

The tubular member 111 may have a number of suitable configurations as shown in schematically in FIGS. 18, 20 and 29-33. In the embodiment illustrated in FIG. 18, tubular member 111 comprises a braided tube of wire or ribbon. FIGS. 29 and 30 illustrate another embodiment in which tubular member 111 comprises a length of metal tubing 162, such as hypodermic tubing, having slots. FIG. 29 illustrates the tubular member 111 in its relatively small-dimensioned configuration for introduction and advancement into the patient's body lumen 121, and FIG. 30 depicts a larger, open configuration of the tubular member 111. The slots cut into the wall of the tubing 162 allow expansion of the occlusive device 110 into the open configuration shown in FIG. 30. In FIG. 29, a metal rod, or bar 170 is contained within the tubular member 111. The metal rod or bar 170 is a different metal than that used for the tubular member 111. The expanded tubular member 111 in FIG. 30 is shown with fiber elements 117 therein, as in FIG. 1, and where the fiber elements 117 within the tubular member 111 are a different metal than that used for the tubular member 111 and a galvanic effect is generated between the different metals.

Likewise, in FIGS. 31 and 32, tubular member 111 is a coil 163 of wire or ribbon. It is obvious that a variety of other suitable configurations may be used for tubular member 111, such as a number of closed sinusoidal rings of wire or ribbon. In FIGS. 31 and 32, two coils 163, 164 and 165 made of metal different from each may form the galvanic effect. In FIG. 31, the coils 163 and 164 are interspersed with each other (one may be “screwed into” the other), or as in FIG. 32, one coil 165 may be contained within the other coil 163.

In still other embodiments, mechanical, adhesive or other anchoring means may be employed to secure the expanded tubular member 111 to the vessel wall defining the body lumen 121. For example, the means to secure a stent or prosthetic device to an aortic or arterial wall described in U.S. Pat. No. 4,140,126; U.S. Pat. No. 4,562,596; U.S. Pat. No. 4,577,631; U.S. Pat. No. 4,787,899; U.S. Pat. No. 5,104,399; U.S. Pat. No. 5,167,614; U.S. Pat. No. 5,275,622; U.S. Pat. No. 5,456,713; and U.S. Pat. No. 5,489,295 may be used with the present invention to interconnect the wall of a patient's body lumen 121 and the tubular member 111.

Some acceptable metals may include: stainless steel, super elastic or shape memory material such as a nickel-titanium (NiTi) alloy such as NITINOL, platinum, tantalum, copper, and gold. In a presently preferred embodiment, the tubular member 111 is a superelatic material, providing a controlled force on the body lumen 121 during expansion of the tubular member 111. The surface of the tubular member's 111 framework may be designed to further facilitate epithelial growth and other tissue ingrowth, as by providing the tubular member 111 with an open or lattice-like framework to promote epithelial growth into as well as around the tubular member 111 to ensure secure attachment to, and embodiment within the wall of the body lumen 121. Suitable surface techniques include EDM machining, laser drilling, photo etching, sintering and the like. Additionally, increasing the surface area of the tubular member 111 can also provide greater adhesion for the epithelial tissue. Suitable surface treatments include plasma etching, sand blasting, machining and other treatments to roughen the surface. In other embodiments, the occlusive device 110 may be coated or seeded to spur epithelialization. For example, the device 110 can be coated with a polymer having impregnated therein a drug, enzyme or protein for inducing or promoting epithelial tissue growth. In yet another refinement, at least part of the device 110, as for example the tubular member 111 or the mesh member 115, could be plated with or otherwise incorporate an inflammatory material to produce an inflammatory response in the tissue of the wall defining the body lumen 121, which further contributes to the obstruction of the lumen 121. For example, the mesh member 115 or mesh layer 116 may incorporate strands or particles of inflammatory material therein. In one embodiment the inflammatory material comprises copper or copper alloy. Other inflammatory materials, such as radioactive materials, may be suitable as well. For example, at least a part of the device 110, as for example the tubular member 111, could be radioactive, emitting alpha, beta or gamma particles.

Because tissue healing and the inflammatory reaction to tissue injury involve cell division and cell growth, it is advantageous in some circumstances to irritate, scrape or “injure” the tissue of a lumen wall surrounding an occlusive device 110 of the invention. This may be accomplished with a denuding fiber bundle on the outer surface of the tip of the catheter placing the occlusive device 110. In such a case, it may denude the fallopian tube or vessel wall or otherwise irritate the tissue in the region where the occlusive device 110 is placed.

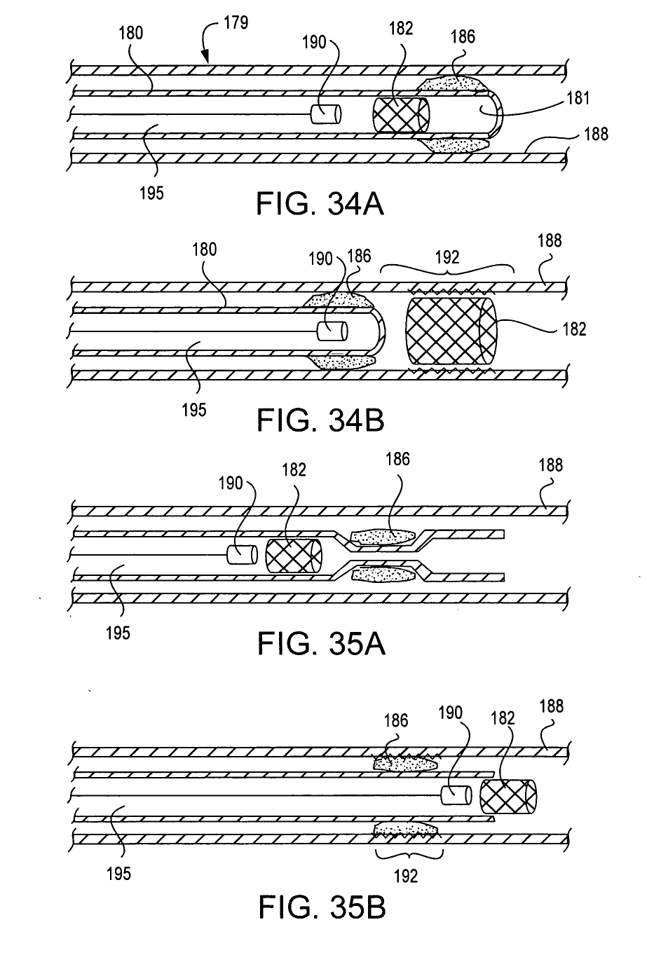

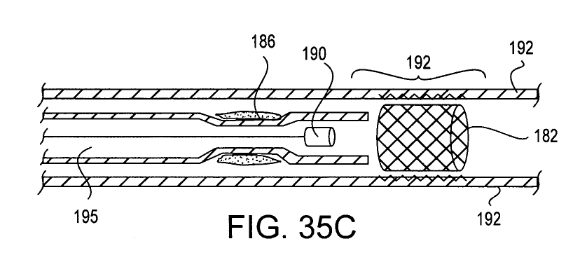

Referring to FIGS. 34A-35C, a delivery catheter 180 for the delivery of an occlusive device 182 into a body lumen 179 is provided with a tissue abrading distal end 181. An abrasive portion 186 on the outer wall of the distal end 181 portion of the catheter 180 is provided, either by building the catheter 180, at least in part, from a material that is abrasive to the wall 188 of the body lumen 179, or by affixing some abrasive material to the outer surface of the catheter 180 at this location.

As the pusher 190 is held firmly in place within the catheter's delivery lumen 195, and the catheter 180 withdrawn, the occlusive device 182 is positioned in the body lumen 179. Because the distal end 181 has an abrasive portion 186, the lumen wall 188 is scrapped or irritated 192, which enhances subsequent tissue ingrowth.

It may be desirable for the catheter 180 outer surface to be smooth and not abrasive where it comes into contact with the lumen wall 188 until the operator desires to scratch the wall 188 surface. For example, if a delivery catheter 180 is being navigated into a body passageway such as a fallopian tube, the operator may not wish to have an abrasive catheter until he desires to deliver the occlusive device 182. In such a case, the abrasive portion 186 of the catheter 180 may be inset in a retracted or navigating configuration (FIG. 35A) and then be expanded into an expanded or abrading configuration (FIG. 35B) by expanding a balloon beneath the abrasive portion 186, or otherwise forcing the abrasive portion 186 outward. In fact, as seen in FIGS. 35A and 35B, if the abrasive portion 186 is naturally biased inward, and is forced outward by the movement of the catheter 180 back around the occlusive device 182, it will naturally abrade the lumen wall 188 for approximately the length of the occlusive device 182. The occlusive device 182 will be laid down in out of the catheter's delivery lumen 195 and expand into contact with the wall 188 of the body lumen 179 approximately along the portion of the wall scrapped or abraded by the catheter 180. As the catheter 180 is further withdrawn, as shown in FIG. 35C, the inward bias of that portion of the catheter 180 previously forced outward against the vessel wall 188 withdraws the abrasive portion 186 so that it is no longer in contact with the vessel wall 188.

Although fibers are illustrated, any mechanical means may be employed to denude or “damage” the vessel wall 188. For instance, fine granules of sand, silica, diamond dust, metal filings o the like may be impregnated onto the distal end 181 of the catheter 180. Similarly, the distal end 181 of the catheter 180 may be roughened so that the surface itself forms an abrasive texture for the abrasive portion 186. The abrasive portion scrapes off lining on delivery and/or removal of the catheter 180, thus causing “injury” to the wall 188 of the body lumen 179, and starting the epithelialization or endothelialization of the vessel at that location to cause ingrowth into or onto the occlusive device 182. The occlusive device 182 could be of a self-expanding nature that is deployed in the region of the body lumen 179 that is scraped or similarly “damaged” by the catheter 180.

The abrasive portion 186 may not be exposed to the tissue of the body lumen 179 wall 188 until desired. Referring again to FIG. 35C, the abrasive portion 186 may be recessed somewhat but expanded when the device 182 is in place to contact and abrade the surface of the lumen wall 188. One example of this would be a balloon, slightly smaller in diameter than the catheter 180 immediately adjacent the balloon and the balloon could be inflated when in place to push the abrasive portion 186 against the tissue at that location. Similarly mechanical expansion such as a guide wire may expand the surface into contact with the wall 188 of the body lumen 179. The expansion illustrated in FIG. 35C shows the expansion of the abrasive portion 186 to a diameter greater than the diameter of other locations along the delivery catheter 180, but merely expanding the abrasive portion 186 to the same diameter as the outer diameter of the rest of the catheter 180 may be sufficient to place the abrasive portion 186 in contact with the lumen wall 188.

The placement of the restricted occlusive device 182 within the section of the delivery catheter 180 having an abrasive portion 186 may expand that portion of the catheter 180 sufficiently to place the abrasive portion 186 in contact with the body lumen wall 188. As the catheter 180 is withdrawn, leaving the occlusive device 182 behind, it abrades the body lumen wall 188 at the location where the occlusive device 182 is delivered.

In addition to or as an alternative to an expanding occlusive device 182, an inner tissue growth-supporting matrix may be provided that is expandable. An example of an expandable inner tissue growth-supporting matrix is a bundle of fibers with a self or balloon expandable frame that springs open when released from the delivery catheter. The fibers could be wrapped around the frame so that the inner matrix also expands with the frame. The frame itself, though, is not a requirement for the concept. The expandable matrix may be strong enough to expand and secure itself to the inner walls of the vessel or conduit such as the fallopian tubes into which it is placed. For example, if contraception or sterilization were desired, the device would be laced at least in part within the fallopian tube or vas deferens. The expandable matrix may also incorporate a very tight woven mesh or closed cell foam that would immediately seal the passageway upon deployment. The matrix may be bundled fibers, woven mesh, sponge-like foam or collagen, metal fibers as in steel wool or metal fiber pads, or gas expandable foam that expands when released from pressure as in a sprayable foam, or any combination of the above materials. The matrix, whether by itself or within a frame, would be capable of expansion within the passageway either by self-expanding mechanism or balloon or other mechanical expansion. The matrix might also have structure or other characteristics that enhance sealing itself to the wall of the vessel or passageway.

In some situations, a device implanted into a body lumen may be subject to forces that would tend to expel the device. This is especially true immediately after placement before the cells of the lumen wall have grown into the occlusive device and helped to secure it to the wall. For example, if an object is placed in the fallopian tubes, the cilia therein may tend to “sweep” the object out of the fallopian tube. Likewise, blood flowing within a vessel may tend to dislodge the device and move it away from the location it was originally placed. This tendency to be expelled or moved is, of course, resisted by the friction of the occlusive device with the walls of the lumen in question. However, if the entire device is unitary and rigid, the force acting to move or expel the device may be sufficient to overcome the friction of the lumen walls with the surface of the device.

If the occlusive device is segmented so that longitudinal force acting on one segment is not transmitted to the adjacent segment, the tendency of the occlusive device to be longitudinally displaced and thereby expelled from the location where it was placed can be greatly reduced.

This desirable characteristic may be obtained by having an occlusive device formed of multiple segments with force absorbing or otherwise non-rigid connecting elements. For example, a multi-segment expandable stent as shown in FIG. 47 may be placed into a lumen with space between the segments. They are butted up against one another within the delivery catheter, but are pushed out of the catheter along a length of body lumen such as the fallopian tube 68 that is longer than the length of catheter lumen they occupied before they were expelled from the catheter. This results in space between the segments, so that they are flexible to undulating motion and do not transmit force from one unit to the other.

In another embodiment, a stent expands into a configuration in which links within the stent are connected by non-rigid links. The stent is lightly held in longitudinal configuration within the delivery catheter. Once the outer sheath is retracted (i.e. once the stent is extruded from the catheter lumen) the stent expands, and the segments are held in lose proximity to one another by flexible links. For instance, the links could be made of polyester fibers that keep the overall device very flexible. Alternatively the links could be made of the same material as the stent frame, allowing all the segments and links to be made from a single piece of tubing, but the connecting members could be further processed, for example by stamping a tube thinner and flatter than the tubular frame elements. These flexible links allow for longitudinal force such as cilia sweeping motion to be applied to one unit without being transmitted to the adjacent unit. The occlusive device could incorporate a tissue growth-supporting member within the lumen of the occlusive device to enhance ingrowth. The growth-supporting member, such as a bundle of fiber, may or may not be segmented, as is the outer portion of the occlusive device. While some advantage may be obtained by segmenting the growth supporting matrix, since it may not be in direct contact with the lumen wall until the cells of the wall grown into the occlusive device, it may not be subjected to the same expulsion forces, such as the sweeping motion of the cilia of the surface of the fallopian tubes.

Where a device is loaded into a placement catheter, as is the case with the occlusive devices here, there is often a significant period of shelf time after the device is prepared and before it is used. This may result in the occlusive device having an undesirable amount of static friction when the user attempts to deploy the device. This is particularly true if the occlusive device is a self-expanding device and may enlarge to snugly fit into the delivery lumen. The result may be that, if unrelieved, the static friction (sometimes colorfully called “stiction”) may cause the operator to apply too much force and expel the occlusive device early or at an inappropriate location, or to direct his attention to forcing the device to be expelled and to accidentally move the catheter to an undesired location. Some device for relieving the stiction would be desirable. The present invention provides such a device.

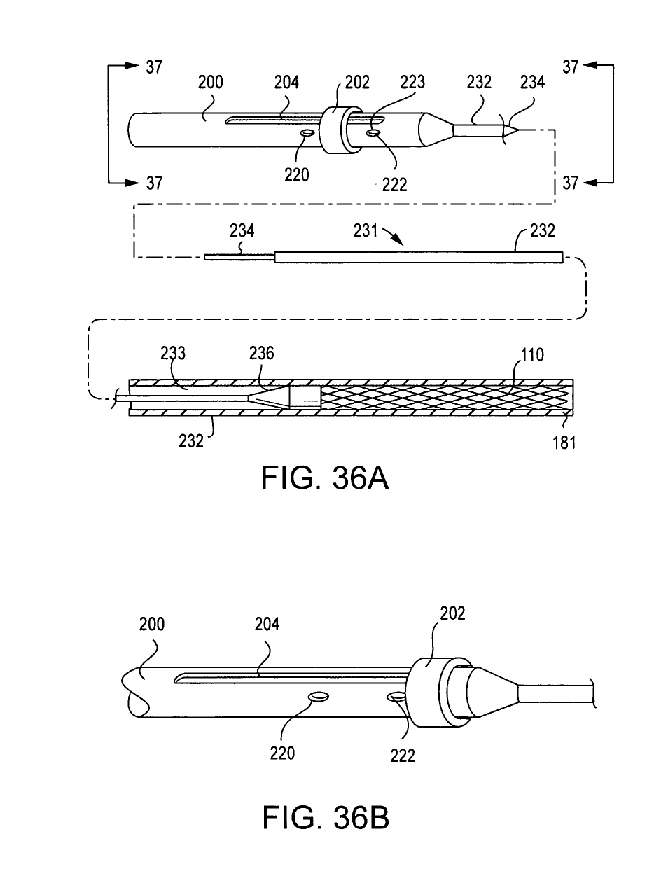

One embodiment of such a device is shown in FIGS. 36A-38. In this embodiment, the self-expanding occlusive device 110 is loaded into the delivery lumen 233 of the delivery catheter 231. Since the occlusive device 110 may be self-expanding against the wall of the body lumen, and may be configured to attach firmly to the wall when positioned within the body lumen and to resist lateral movement within the body lumen, placement generally will not be optimum if the device 110 is pushed out the end of the delivery catheter 231 and then further pushed longitudinally along the lumen wall.

As depicted in FIGS. 36A and 36B, the delivery catheter 231 has a handle 200, a catheter shaft 232 a pushing wire 234, a pushing element 236 and an occlusive device 110. The handle 200 includes a slidable ring 202, a slot 204 that serves as a track within which the slidable ring 202 will slide forward or back, and a fixation point 206 where the pushing wire 234 is rigidly attached to the handle 200. The pushing element 236 is also fixed to the pushing wire 234 so that imparting longitudinal, motion to the pushing wire 234 will longitudinally displace the pushing element 236 relative to the catheter 231.

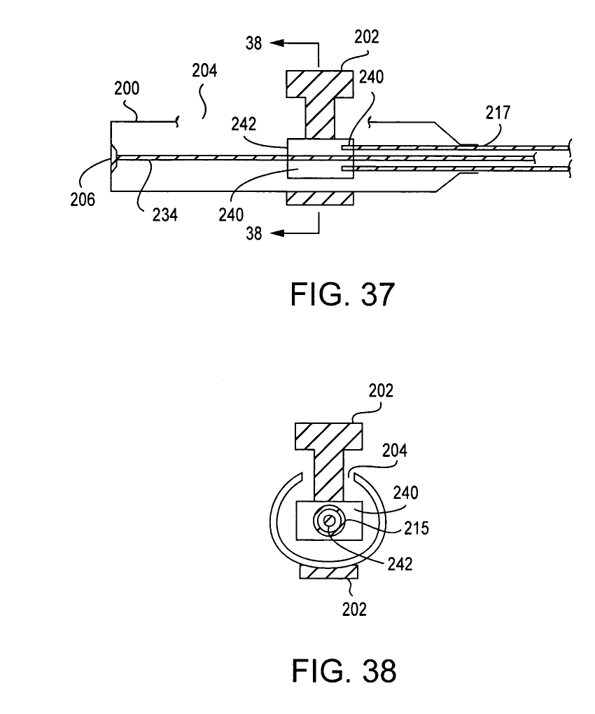

Referring to FIGS. 37 and 38, the slidable ring 202 is attached to a block 240. The block 240 has a bore 242 through the middle of the block 240, and the pushing wire 234 goes through the bore 242 and is attached to the end of the handle 200 at fixation point 206. The proximal end of the catheter 231 is embedded in the block 240. The opening 217 of the handle 200 acts as a bearing and permits the smooth longitudinal movement of the catheter 231 therethrough.

Surface features are provided to restrict the longitudinal motion of a sliding element, such as the slidable ring 202. In FIG. 36B, the surface features are in the form of a first set screw 220 and a second set screw 222 attached to the handle 200 to restrain the longitudinal movement of the slidable ring 202. As shown in FIG. 36B, both set screws 220 and 222 are in place and snugly restrain the slidable ring 202 from any longitudinal motion in the far forward position. In preparation for placement of the occlusive device 110, the first setscrew 220 is removed and the slidable ring 202 pulled back a short distance, as restrained by the second setscrew 222 still in place. This has the effect of moving the occlusive device 110 into place for deployment and breaking any stiction that may have developed during storage.

When final deployment is desired, the second setscrew 222 is removed and the slidable ring 202 may be pulled all the way back, expelling the occlusive device 110 from the delivery lumen of the catheter 233 and depositing it in the body lumen.

It should be understood that, although in the illustrations here the slidable ring 202 is configured for easy griping and manual sliding, other mechanisms are anticipated. For example, a trigger mechanism (not shown) could be employed to gently and controllably pull the ring 202 back relative to the handle 200 and thus to pull the catheter 231 back relative to the occlusive device 110.

The proper location of the distal end 181 of the catheter 231 containing the occlusive device 110 may be determined by direct observation, as with a multi-colored catheter shaft 232 described elsewhere in this application, or with ultrasound, or x-ray, or fluoroscopy or other suitable means.

It will be appreciated that the use of multiple safety set 220 and 222 screws may be used to control the deployment of multiple occlusive devices 110 using the same delivery catheter 231. After removal of the first anti-deployment safety set screw 220, the slidable ring 202 may be moved proximally until it encounters the second anti-deployment set screw 222. This is precisely the amount needed to deploy a first occlusive device 110 and to set a second occlusive device at the distal end 181 within the delivery lumen 233. The delivery catheter 231 is then retracted in the next body lumen, for example the other fallopian tube of a patient undergoing bilateral tubal occlusion, and the second anti-deployment screw 222 is removed and the slidable ring 202 moved further proximal down the handle 202, deploying the second occlusive device.

It should be noted that this delivery device 231 could be used to deliver occlusive devices 110, carotid stents, AAA (abdominal aortic aneurysm) stent grafts, vena cava filters, embolic protection device, or fallopian tube occlusion device for permanent or reversible sterilization.

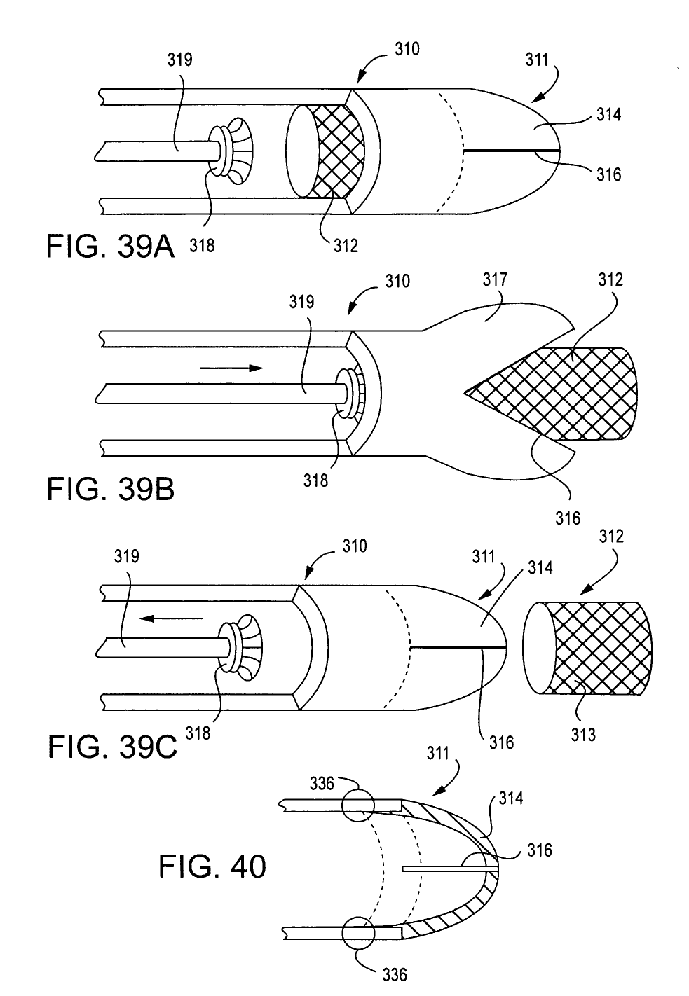

Referring to FIGS. 39-42, a soft, rounded tip portion 314 comprised of silicone rubber or latex can be attached to the distal end 311 of the delivery catheter 310. The rounded tip portion 314 has a split 316 so that, when an occlusive device 212 is pushed out of the delivery catheter 310, the rounded tip portion 314 opens up to allow the exit of the occlusive device 312, and then closes back up when the device 312 has been expelled. This has the advantage of allowing a delivery catheter 310 to be maneuvered through a body lumen, for example a blood vessel or bile tract or fallopian tube, to deliver an occlusive device 312, while remaining closed to the body fluid through which it is maneuvered.

The rounded tip portion 314 should be movable between an open configuration and a closed configuration by the act of expelling the occlusive device 312. The rounded tip portion 314 should be biased to a shut position so that it automatically returns to the shut position when the force causing it to open is removed.

Referring again to FIGS. 39A-39C, the delivery catheter 310 is depicted loaded with an occlusive device 312 such as self-expanding stent 313. A pusher 318 transmits force to push the stent 313 out of the delivery catheter 310. The force may be transmitted from the proximal end of the catheter (not shown) through a pusher wire 319. When the force is applied, as shown in FIG. 39B, the stent 313 pushes against the interior of the distal end 311 of the catheter 310, forces the sides of the distal end 311 apart along the split 316 and simultaneously forces the stent 313 out of the distal end 311 of the catheter 310 between the two end portions 317 of the distal end 311. When the stent 313 is fully expelled, and the pusher 318 is withdrawn, as shown in FIG. 39C, the two end portions 317 of the distal end 311 return to the closed position and form a closed end to the delivery catheter 310.

The stent 313 may be an expandable device, and may be expelled in a first, compressed configuration and may expand, either by some force such as a balloon expansion, or by self expansion such as might occur in a stent made of Nitinol.

The rounded tip portion 314 may be separately manufactured from a suitable material and joined to the end of a delivery catheter 310. Referring to FIG. 40, a catheter 310 may be made of one material, for example PET and a rounded tip portion 314 may be made of a suitably elastic material such as such as low durometer PEBAX, silicone rubber or polyurethane. The rounded tip portion 314 is bonded to the catheter 310 by a suitable adhesive such as UV activated CA.

To facilitate pushing the stent 313 out of the catheter 310, the transition 336 between the rounded tip portion 314 and the catheter 310 should be smooth. This can be accomplished in several ways. A mandrel can be inserted into the bonded portion by stretching the rounded tip portion 314 apart over the mandrel, and an abrasive portion on the mandrel can polish the transition 336. Similarly, a mandrel with a heated portion can be used to smooth the transition 336. Alternatively, a lubricious substance, for example Teflon, can be laid down over the transition 336 to allow smooth pushablility.

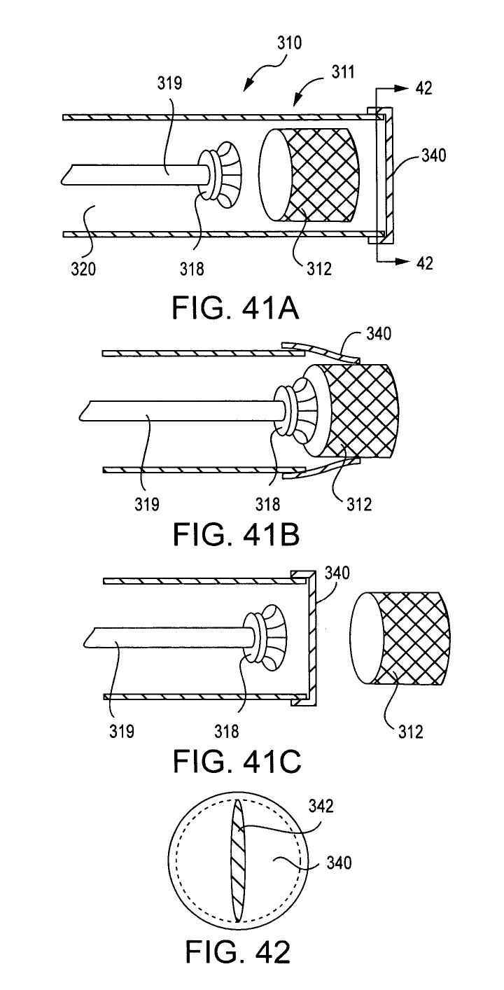

The round tip portion 314 need not be rounded. If the distal end 311 of the catheter 310 is likely to be pushed into an elongate passageway such as a vessel or a tube (e.g. fallopian tube) then the rounded tip portion 314 would be advantageous. However, referring to FIGS. 41A-42, in an alternative embodiment, a flat elastic membrane 340 is stretched across the distal end 311 of the catheter lumen 320. A slit 342 as depicted in FIG. 42 across the membrane 340 allows a stent 313 to be pushed out the distal end 311 of the catheter 310. The slit 342 in the membrane 340 will expand, allow the passage of the stent 313, and then when the pusher 318 is withdrawn, will close and provide a seal for the distal end 311 of the catheter lumen 320.

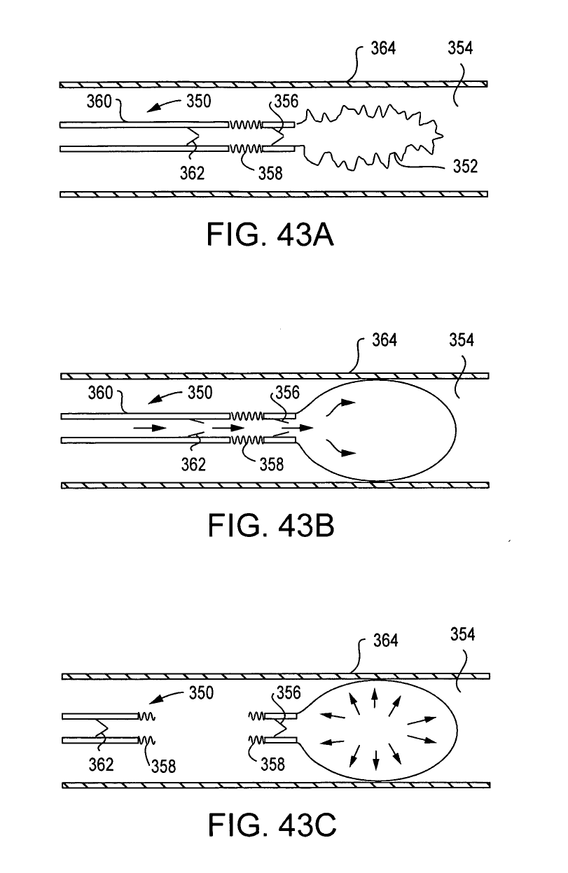

Referring now to FIGS. 43A-43C, another embodiment of the present invention includes the use a balloon-like barrier device 352 for providing immediate occlusion of a body lumen 354 when placing an occlusive device in the body lumen 354. A delivery catheter 350 having an uninflated balloon 352 attached to its distal tip is placed into a body lumen or passageway 354 such as a fallopian tube or a blood vessel. The balloon 352 is attached distal of a one-way valve 356 between the balloon 352 and the catheter 350. A detachable valve or region 358 is located between the catheter shaft 360 and the one-way valve 356. A second one-way valve 362 acts as a back flow valve within the body of the catheter 350.

When deployed, as illustrated in FIGS. 43A-43C, the catheter 350 with the deflated balloon 352 is advanced to the desired location within the body lumen 354, inflation fluid (indicated by the arrows in FIGS. 43B and 43C) is introduced through the catheter 350 into the balloon 352, inflating the balloon 352 so that it is firmly in contact with the body lumen walls 364. The detachable section 358 which may be a detachment valve or a very breakable portion of the catheter wall which is easily severed is then severed and the catheter 350 is withdrawn. The second one-way valve, or back-flow valve 362, if present prevents any fluid in the body lumen 354 from traveling back up the catheter 350. The one-way valve 356 closes and preserves the balloon in the inflated condition.

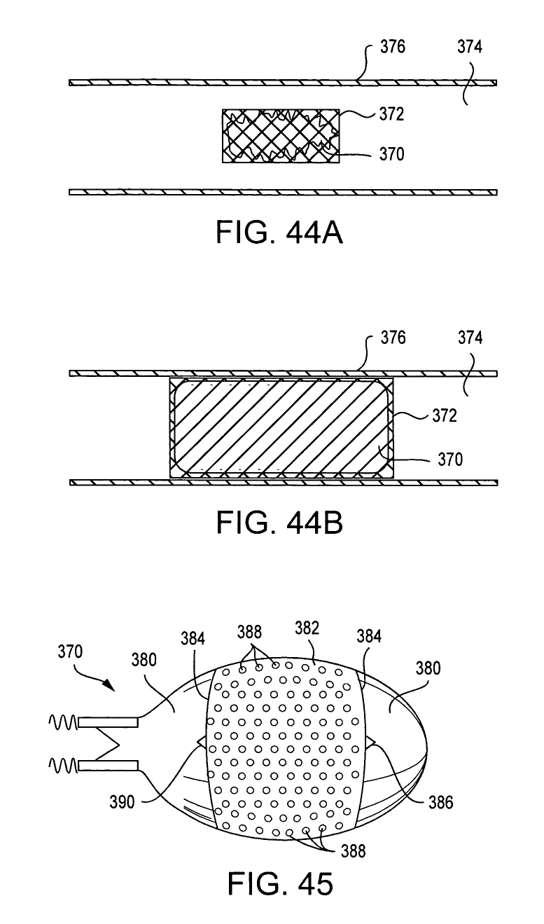

An alternative method of deploying an impermeable barrier to attain immediately effective closure of a body lumen is illustrated in FIGS. 44A-44B. A balloon 370 is mounted within a self-expanding stent 372. The stent 372 is delivered to a desired location within the body lumen 374 and released as previously described. When the stent 372 expands so that it is in contact with the lumen walls 376, the balloon 370 is expanded, which then forms a barrier.

As illustrated in FIG. 45, the balloon 370 may be formed in multiple segments. For example, end segments 380 of the balloon 370 may be impermeable material such as PET or silicone rubber. An intermediate segment 382 may be permeable, for example it might contain track etched pores 388 to make the material slightly porous, so that tissue ingrowth may occur. The segments 380, 382 are separately inflatable and separated by sealing walls 384. The walls 384 are separated by one way valves 386 and pressure valves 390 so that once the pressure reaches a desired level, inflation fluid will move from one end segment 380 to inflate the intermediate segment 382 and the second end segment 380. When the pressure is released, the pressure valve 390 will close, the intermediate segment 382 will lose pressure and no fluid will pass from the inflated end segments 380 to the intermediate segment 382. In this manner, the two end segments 380 will be inflated barriers for immediate effectiveness, and the intermediate segment 382 will be a porous section supporting tissue ingrowth.

The balloon 380 is constructed of any material that is medically acceptable, for example PET. PET is also an advantageous material that may be treated to create a porous section. For example, for a balloon 370 with two end segments 380 and an intermediate segment 382, the two end segments 380 may be shielded and the intermediate segment 382 etched to create a section that is porous enough to permit tissue ingrowth. Alternatively, bio-absorbable material may be used for the balloon 370, but the rate of bio-absorption must be sufficiently slow that the tissue ingrowth matrix will not be absorbed prior to effective sealing of the body lumen 374 by tissue ingrowth.

In one embodiment, the balloon 370 is inflated by any biocompatible material, for example saline. Alternatively, an inflation medium such as expandable foam may be used, but care must be taken that the intermediate segment 382 be sufficiently permeable to permit effective tissue ingrowth.

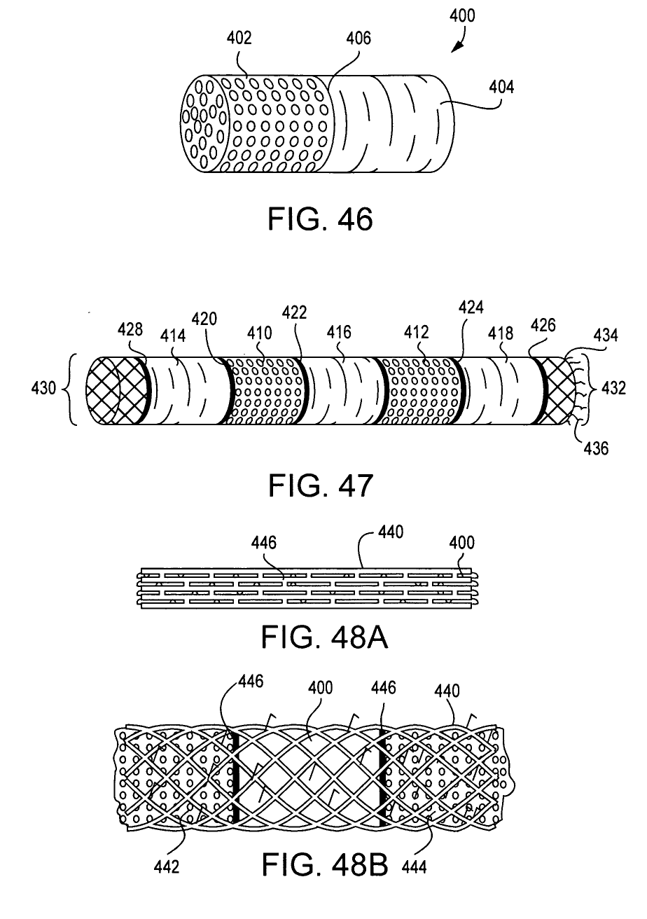

Referring to FIGS. 46-47, in another embodiment the occlusive device is in the form of a foam plug 400. The plug may be comprised of segments. For example, a first segment 402 may be comprised of open celled foam and a second segment 404 may be comprised of solid material that is compressible or compressible closed cell foam. The segments 402, 404 are separated by a membrane 406, or formed in alternating sections.

The plug 400 is inserted into the distal tip of the delivery catheter by compressing the plug 400 and placing it in the delivery lumen. This may be done by pushing the plug 400 through a funnel into the delivery lumen. The funnel may have a highly lubricious interior surface to facilitate the compression and movement of the plug 400 into the catheter delivery lumen.

The plug 400 may have various configurations. It may comprise multiple segments as show on in FIG. 47. The segments may alternate between permeable open cell segments 410, 412 that permit and may even enhance tissue ingrowth, and closed cell segments 414, 416, 418 and may be divided by membranes 420, 422, 424. There may also be membranes 426, 428 at the ends of the plug. There may in some embodiments be short stent-like portions 430, 432 at the ends of the plug 400 to anchor the plug 400 in place once it has been place and to resist expulsion by the body lumen, e.g. the cilia of the fallopian tube. The short stent-like portions 430, 432 may have projecting wires 434, 436 to firmly attach the plug 400 to the surface of the lumen wall.

In an alternative embodiment, (see FIGS. 48A and 48B) the foam plug 400 may be encased in a stent-like structure 440. The plug 400 could take any of the forms as discussed above, for example the plug 400 may contain permeable open cell segments 442, 444 separated by membranes 446 and be contained within the stent-like structure 440 either in place of or in addition to fibers that encourage tissue ingrowth. The stent-like structure 440 and the foam plug 400 are both shown in FIG. 48A in their unexpanded configuration, and they may expand to an expanded configuration as shown in FIG. 48B. The pressure of the foam plug 400 to expand may assist in expanding the stent-like structure 440 or the stent-like structure 440 may be self-expanding as described above. The plug 400 may be preformed and inserted into the stent-like structure 440, or may be formed in the stent-like structure 440, for example by injecting the plug 400 into a stent-like structure 440 and after the plug 400 has dried and formed, compressing the stent-like structure 440.

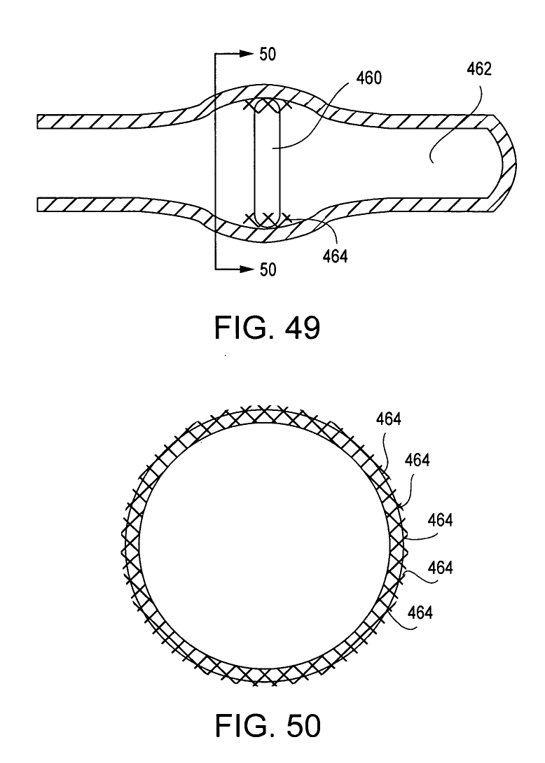

Referring now to FIGS. 49 and 50, an immediately effect barrier device may take the form of a disc 460 inserted into the body lumen 462. The disc 460 may have projections 464, 466, such as wires, or a short length of stent-like structure, to aid in positioning and securing the disc 460, and/or to enhance tissue ingrowth. The disc 460 may be slightly elongate to create a slightly cylindrical shape to enhance the placement and ensure that the disc 460 does not rotate into a flat position that does not seal the body lumen 462.

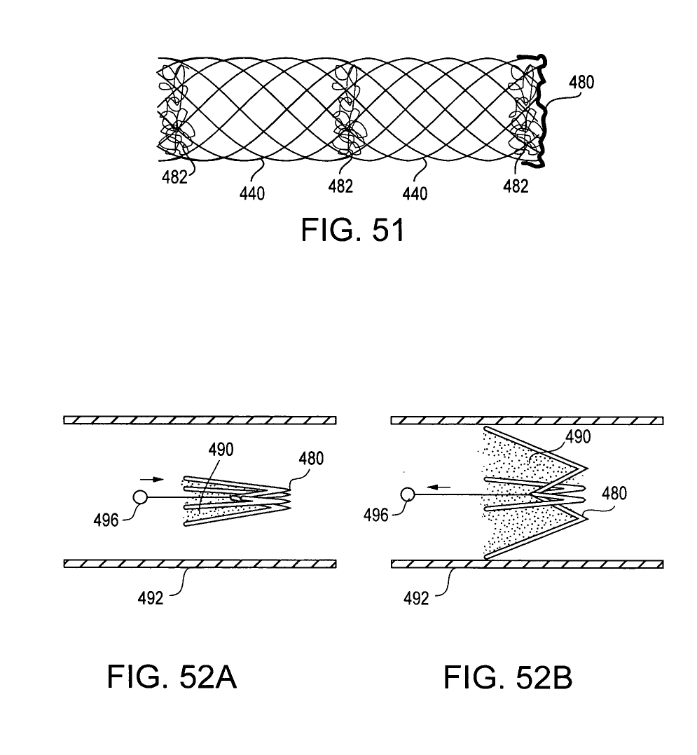

Referring to FIG. 51, an impermeable membrane 480 stretched over at least one end of an expandable stent-like structure 440 may provide for immediate effectiveness of sealing a body lumen. The impermeable membrane 480 may be bioabsorbible and absorb slowly enough that tissue ingrowth permanently closes the body lumen by the time that the membrane 480 degrades such that the seal is no longer intact. Tissue ingrowth may be encouraged in any of the previously described methods, for example by providing fibers 482 within the structure of the stent-like structure 440.

Referring to FIGS. 52A and 52B, the membrane 480 may be stretched across deployable structures in the nature of umbrella covering. The device 490 may be in retracted configuration as shown in FIG. 52A to provide for insertion, then deployed, as shown in 52B to provide immediate effective sealing against the walls 492 of the body lumen. An activation device 496 may be manipulated to change the occlusive device 490 from a collapsed configuration to an expanded configuration. In this way the device 490 may be manipulated after placement, and perhaps a significant time after placement, to collapse it from expanded back to collapsed configuration to provide for removal of the occlusive device 490.

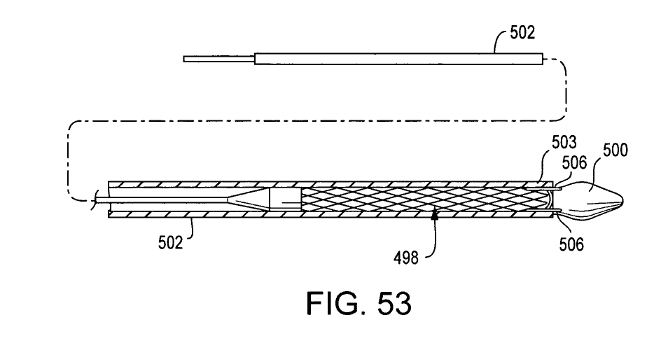

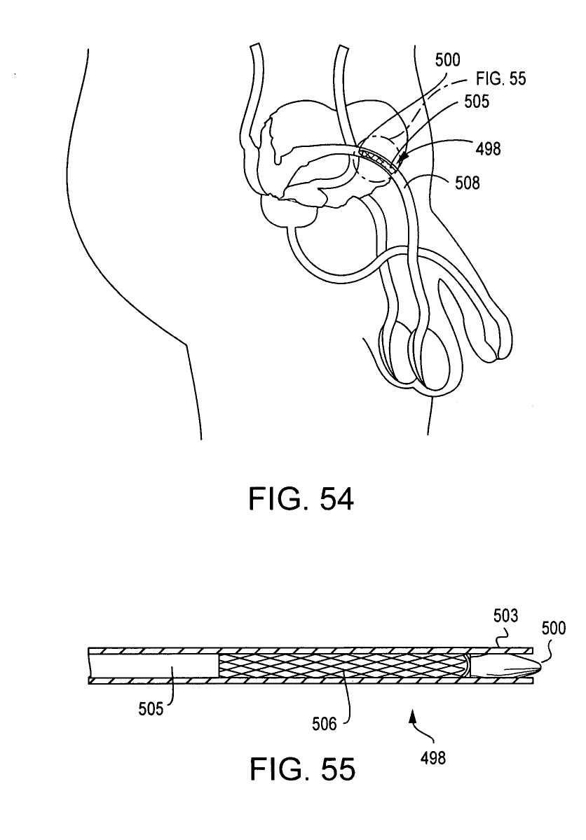

Another embodiment for an immediately effective occlusive device 490 is shown in FIGS. 53-55. A bulbous impermeable end 500 is placed onto the distal end 503 of a delivery catheter 502. The bulbous impermeable end 500 is then placed into a body lumen 508, dislodged from the end of the delivery catheter 502, and left in place.

In one embodiment, when the bulbous impermeable end 500 is placed into the vas 505 of a male patient, FIGS. 55-56, it will effectively obstruct the vas 505 to prevent the passage of sperm and thereby create the desired sterility. In that instance, however, it will not prevent sperm already downstream of the device 498 from being expelled in seminal fluid and, in the same manner as a vasectomy, some additional birth control will be necessary for a short time after placement of the occlusive device 498, but it would not be necessary to wait the full time that it would take for tissue ingrowth to occur to fully occlude the vas 505.

Additional structural elements could be added to provide for tissue ingrowth to obstruct the body lumen 508. In the case of the vas 505, a stent-like structure on either or both sides of the bulbous device could create a scaffold for tissue ingrowth. In the case of female sterility, the stent-like structure could be placed on the distal end of the bulbous impermeable end 500 so that the fallopian tubes would be sealed over time by tissue ingrowth into the stent-like structure, and immediately effective sealing of the fallopian tubes would occur by the placement of the bulbous impermeable end 500 into the ostium and anchoring it in the muscular tissue of the uterus.

It will be appreciated by those skilled in the art that various modifications, additions, deletions, combinations and changes may be made to the examples described here-above and shown in the drawings, without departing from the intended spirit and scope of this invention. All such reasonable modifications, additions, deletions, combinations and changes are included in this disclosure.

What is claimed:

1. An occlusive device, said device comprising:
a first element constructed of a first metallic material; and
a second element constructed of a second metallic material, said first metallic material is different than said second metallic material, said first and second elements being electrically separated from each other along at least a significant portion of said elements.

2. An occlusive device as recited in claim 1 wherein said device comprises a stent and said elements comprise wires that form said combine to form at least part of said stent.

3. A stent as in claim 2 wherein at least one of said wires is gold.

4. A stent as in claim 2 wherein at least one of said wires contains iron.

5. An occlusive device as in claim 1 wherein said first element is contained within said second element.

6. An occlusive device as in claim 1 wherein at least one of said metallic elements is a helically shaped spring.

7. An occlusive device as in claim 6 wherein at least two of said elements are in the form of a helically shaped spring.

8. An occlusive device as in claim 6 wherein said first element is a helically shaped spring and said second element is a helically shaped spring, said first element being contained within said second element.

9. An occlusive device as in claim 6 wherein said first element is a helically shaped spring and said second element is a helically shaped spring, the coils of said first element and the coils of said second element being interspersed with each other.

10. An occlusive device as in claim 1 wherein the two metal elements are in the form of metal sheets coiled within each other.

11. An occlusive device for generating a galvanic action, said occlusive device comprising an elongate cylindrical, said device having metallic granules interspersed on or in said elongate cylindrical structure, said granules comprising at least two different metals.

12. A delivery catheter system for delivering an occlusive device into a body lumen comprising:
a tissue abrading distal tip portion comprising an abrasive portion of the outer wall of the distal end of the catheter; and
a pusher device for holding the occlusive device relative stationary in a longitudinal direction while the catheter is withdrawn which acts to expel the occlusive device from the catheter delivery lumen.

13. A delivery catheter system as in claim 12 further comprising a catheter region that has a first, retracted configuration and a second, expanded configuration, whereby the expanded configuration brings the abrasive region into contact with the lumen wall, and the retracted configuration withdraws the abrasive portion away from contact with the vessel wall.

14. A delivery catheter system as in claim 12 where the act of expelling the occlusive device acts to change the delivery catheter from the first configuration to the second configuration.

15. A delivery catheter system as in claim 12 further wherein when the occlusive device is fully expelled into the body lumen, the catheter returns to said first configuration.

16. A delivery catheter system as in claim 13 wherein said catheter outer surface is biased toward said first configuration, and requires application of force to move said catheter outer surface to said second configuration.

17. A delivery catheter system as in claim 14 wherein said abrasive out surface is elastically biased to said first configuration.

18. A delivery catheter system as in claim 14 where the catheter is changed from said first configuration to said second configuration by the occlusive device moving through said delivery lumen toward the open distal tip of said catheter.

19. A delivery catheter system as in claim 14 where the catheter is changed from said first configuration to said second configuration by the inflation of a balloon beneath the abrasive surface.

20. A delivery catheter system for delivering an occlusive device, said system comprising:
a catheter shaft;
a pusher wire contained within at least a portion of said catheter shaft, said pusher wire fixedly attached to said catheter handle; and
a handle having a sliding member and a catheter clasping member, said catheter clasping member attached to said sliding handle and to said catheter shaft such that moving said sliding member relative to said catheter handle serves to move said catheter shaft relative to said pusher wire.

21. A delivery catheter system as in claim 20 wherein said handle has an outer surface, and said slidable member slides along said outer surface, said outer surface comprising a surface feature that prevents sliding motion of said slidable member past said surface feature.

22. A delivery catheter system as in claim 21 where said surface feature is the head of a removable set screw.

23. A delivery catheter system as in claim 21 further comprising two set screws, wherein said slidalbe handle has a range of sliding motion, and said handle contains at least two surface features, said surface features located at different positions along said sliding range.

24. A delivery catheter system as in claim 20 further wherein said pusher wire is attached to a pusher element, said pusher element contained within a delivery lumen of said catheter near the distal end of said catheter, said delivery lumen further containing an occlusive device between said pusher element and said distal end of said delivery lumen, the proximal end of said occlusive device sized so that when the pusher element is moved longitudinally in said delivery lumen and contacts the proximal end of said occlusive device, it moves said occlusive device the same distance longitudinally as the pusher wire is moved after said contact thereby causing longitudinal movement of said occlusive device approximately equivalent to the longitudinal movement of said pusher wire.

25. A delivery catheter system as in claim 24 wherein a surface feature on the surface of said handle prevents further slideable motion after said slideable member contacts said surface element.

26. A delivery catheter system as in claim 25 wherein said surface feature is a first setscrew.

27. A delivery catheter system as in claim 26 wherein said first set screw is located such that the slideable motion is sufficient to expel the occlusive device out of the distal end of the delivery lumen of said catheter.

28. A delivery catheter system as in claim 27 further comprising a second set screw, said second set screw located so as to allow sufficient longitudinal relative motion between the catheter shaft and the pusher wire to cause the pusher element to move the occlusive device longitudinally toward the distal end of the delivery lumen but not allow sufficient longitudinal relative motion to permit the pusher to move the occlusive device out of the delivery lumen, and said first set screw is located to allow sufficient relative longitudinal motion between the pusher wire and the catheter shaft to allow the pusher to expel the occlusive device out of the distal end of the delivery lumen.

29. A delivery catheter comprising a catheter delivery lumen having a distal cone, said distal cone having a first, closed configuration and a second open configuration, said distal cone biased in the direction of said first, closed configuration, said distal cone being moved from said first closed configuration to said second, open configuration by movement of an occlusive device through said delivery lumen and out the distal end of said lumen, through the distal cone.

30. An occlusive device comprising:
a plurality of segments, said segments joined to the longitudinally adjacent segment by a flexible joining element such that not all the longitudinal force applied to one element is transmitted to the adjacent element.

31. An immediately effective occlusive device, the device comprising:
a barrier device, said barrier device substantially impermeable to the passage of cells within said body lumen; and
a scaffold device, said scaffold device permitting tissue ingrowth from the walls of said body lumen into said scaffold device such that an occlusion of said body lumen occurs when said tissue ingrowth has substantially occurred, and said scaffold device attached to said barrier device such that placement of said scaffold device into said body lumen places said barrier device and positions it in such a manner as to accomplish substantially complete occlusion of said body lumen.

32. An occlusive device as in claim 31 wherein said body lumen is the fallopian tube and said cells are egg cells.

33. An occlusive device as in claim 31 wherein said body lumen is the vas and said cells are sperm cells.

34. An occlusive device as in claim 31 wherein the scaffold is open celled foam and the barrier device is closed cell foam.

35. An occlusive device as in claim 31 wherein the occlusion device is formed of adjacent cylindrical lengths of foam, and the scaffold is open celled foam and the barrier device is closed cell foam.

36. An occlusive device as in claim 35 further comprising impermeable membrane forming the boundary between adjacent segments of said cylindrical foam.
